SEQUENCE LISTING (32 sequences)

SEQ ID NO: 1 - protein, 274 aa, Neisseria meningitidis
MNRTAFCCLSLTTALILTACSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 2 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 3 - protein, 281 aa, Neisseria meningitidis
MNRTAFCCLSLTTALILTACSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 4 - protein, 244 aa, Neisseria meningitidis
MASHNTTHQMKTLCSSCSLRELCLPVGLLPNELSQLDAVIRQSRRLKKGEYLFCVGEAFTSLFAIRSGFFKTTVASQDGRDQVTGFFMSGELIGMDGICSHVHSCDAVALEDSEVCELPFTHIEELGQNIPSLRTHFFRMMSREIVRDQGVMLLLGNMRAEERIAAFLLNLSQRLYSRGFAANDFILRMSREEIGSYLGLKLETVSRTLSKFHQEGLISVEHKHIKILNLQVLKKMVSGCSHAI

SEQ ID NO: 5 - protein, 244 aa, Neisseria meningitidis
MASHNTTHQMKTLCSSCSLRELCLPVGLLPNELSQLDAVIRQSRRLKKGEYLFCVGEAFTSLFAIRSGFFKTTVASQDGRDQVTGFFMSGELIGMDGICSHVHSCDAVALEDSEVCELPFTHIEELGQNIPSLRTHFFRMMSREIVRAQGVMLLLGNMRAEERIAAFLLNLSQRLYSRGFAANDFILRMSREEIGSYLGLKLETVSRTLSKFHQEGLISVEHKHIKILNLQVLKKMVSGCSHAI

SEQ ID NO: 6 - protein, 250 aa, Escherichia coli
MIPEKRIIRRIQSGGCAIHCQDCSISQLCIPFTLNEHELDQLDNIIERKKPIQKGQTLFKAGDELKSLYAIRSGTIKSYTITEQGDEQITGFHLAGDLVGFDAIGSGHHPSFAQALETSMVCEIPFETLDDLSGKMPNLRQQMMRLMSGEIKGDQDMILLLSKKNAEERLAAFIYNLSRRFAQRGFSPREFRLTMTRGDIGNYLGLTVETISRLLGRFQKSGMLAVKGKYITIENNDALAQLAGHTRNVA

SEQ ID NO: 7 - DNA, 26 bp, Artificial sequence
  Immunostimulatory oligonucleotide
ncncncncncncncncncncncncnc

SEQ ID NO: 8 - protein, 11 aa, Artificial sequence
  Cationic peptide
KLKLLLLLKLK

SEQ ID NO: 9 - protein, 644 aa, Neisseria meningitidis
MASPDVKSADTLSKPAAPVVSEKETEAKEDAPQAGSQGQGAPSAQGGQDMAAVSEENTGNGGAAATDKPKNEDEGAQNDMPQNAADTDSLTPNHTPASNMPAGNMENQAPDAGESEQPANQPDMANTADGMQGDDPSAGGENAGNTAAQGTNQAENNQTAGSQNPASSTNPSATNSGGDFGRTNVGNSVVIDGPSQNITLTHCKGDSCSGNNFLDEEVQLKSEFEKLSDADKISNYKKDGKNDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPSKGEMLAGTAVYNGEVLHFHTENGRPSPSRGRFAAKVDFGSKSVDGIIDSGDGLHMGTQKFKAAIDGNGFKGTWTENGGGDVSGKFYGPAGEEVAGKYSYRPTDAEKGGFGVFAGKKEQDGSGGGGATYKVDEYHANARFAIDHFNTSTNVGGFYGLTGSVEFDQAKRDGKIDITIPVANLQSGSQHFTDHLKSADIFDAAQYPDIRFVSTKFNFNGKKLVSVDGNLTMHGKTAPVKLKAEKFNCYQSPMAKTEVCGGDFSTTIDRTKWGVDYLVNVGMTKSVRIDIQIEAAKQ

SEQ ID NO: 10 - protein, 434 aa, Neisseria meningitidis
MVSAVIGSAAVGAKSAVDRRTTGAQTDDNVMALRIETTARSYLRQNNQTKGYTPQISVVGYDRHLLLLGQVATEGEKQFVGQIARSEQAAEGVYNYITVASLPRTAGDIAGDTWNTSKVRATLLGISPATRARVKIVTYGNVTYVMGILTPEEQAQITQKVSTTVGVQKVITLYQNYVQRGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 11 - protein, 350 aa, Neisseria meningitidis
MKHFPSKVLTTAILATFCSGALAATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVG

SEQ ID NO: 12 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYHGKAFGSDDPNGRLHYTIDFAAKQGYGRIEHLKTPEQNVDLAAADIKPDGKRHAVISGSVLYNQAEKGSYSLGIFGGKAQEVAGSAEVKIGEGIRHIGLAAKQ

SEQ ID NO: 13 - protein, 247 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDLGGEHTAFNQLPDGKAEYRGTAFGSDDAGGKLTYTIDFTKKQGNGKIEHLKSPELNVELASAEIKADGKSHAVILGDVRYGSEEKGSYSLGIFGGRAQEVAGSAEVKTVNGIRHIGLAAKQ

SEQ ID NO: 14 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDLGGEHTSFDKLPEGGRATYRGTAFGSDDAGGKLTYTIDFAAKQGNGKIEHLKSPELNVDLAAAEIKADEKSHAVILGDVRYNQAEKGTYSLGIFGGKAQEVAGSAEVKTVNGIRHIGLAAKQ

SEQ ID NO: 15 - DNA, 6 bp, Artificial sequence
  Mutant -10 promoter element
taccgc

SEQ ID NO: 16 - DNA, 14 bp, Artificial Sequence
  putative FNR-box
ttgacnnnnctcat

SEQ ID NO: 17 - DNA, 185 bp, Neisseria meningitidis
ttcccacacctaaaataacattagaaacattatcataaatcggaatatccgaatcccgaaacgtcaaaacccgacaaacctgcatactggcatcgttaatataaaatcaatgagctgtttatggttttttgctgtaaaaaacattataatccgccttatttacctattgcccaaggagacacaaa

SEQ ID NO: 18 - DNA, 157 bp, Neisseria meningitidis
caggttgcctgtaaacaaaatgccgtctgaaccgccgttcggacgacatttgatttttgcttctttgacctgcctcattgatgcggtatgcaaaaaaagataccataaccaaaatgtttatatattatctattctgcgtatgactaggagtaaacct

SEQ ID NO: 19 - DNA, 14 bp, Artificial sequence
  FNR-box consensus
ttgatnnnnatcaa

SEQ ID NO: 20 - DNA, 6 bp, Artificial Sequence
  -10 hexamer
taccat

SEQ ID NO: 21 - DNA, 6 bp, Artificial Sequence
  -10 hexamer
ttgatg

SEQ ID NO: 22 - protein, 180 aa, Neisseria meningitidis
MVSAVIGSAAVGAKSAVDRRTTGAQTDDNVMALRIETTARSYLRQNNQTKGYTPQISVVGYDRHLLLLGQVATEGEKQFVGQIARSEQAAEGVYNYITVASLPRTAGDIAGDTWNTSKVRATLLGISPATRARVKIVTYGNVTYVMGILTPEEQAQITQKVSTTVGVQKVITLYQNYVQR

SEQ ID NO: 23 - protein, 488 aa, Neisseria meningitidis
MFKRSVIAMACIFALSACGGGGGGSPDVKSADTLSKPAAPVVSEKETEAKEDAPQAGSQGQGAPSAQGSQDMAAVSEENTGNGGAVTADNPKNEDEVAQNDMPQNAAGTDSSTPNHTPDPNMLAGNMENQATDAGESSQPANQPDMANAADGMQGDDPSAGGQNAGNTAAQGANQAGNNQAAGSSDPIPASNPAPANGGSNFGRVDLANGVLIDGPSQNITLTHCKGDSCSGNNFLDEEVQLKSEFEKLSDADKISNYKKDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPAKGEMLAGAAVYNGEVLHFHTENGRPYPTRGRFAAKVDFGSKSVDGIIDSGDDLHMGTQKFKAAIDGNGFKGTWTENGSGDVSGKFYGPAGEEVAGKYSYRPTDAEKGGFGVFAGKKEQD

SEQ ID NO: 24 - protein, 364 aa, Neisseria meningitidis
MSMKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 25 - protein, 174 aa, Neisseria meningitidis
MKKALATLIALALPAAALAEGASGFYVQADAAHAKASSSLGSAKGFSPRISAGYRINDLRFAVDYTRYKNYKAPSTDFKLYSIGASAIYDFDTQSPVKPYLGARLSLNRASVDLGGSDSFSQTSIGLGVLTGVSYAVTPNVDLDAGYRYNYIGKVNTVKNVRSGELSAGVRVKF

SEQ ID NO: 26 - protein, 792 aa, Neisseria meningitidis
MKPLQMLPIAALVGSIFGNPVLAADEAATETTPVKAEIKAVRVKGQRNAPAAVERVNLNRIKQEMIRDNKDLVRYSTDVGLSDSGRHQKGFAVRGVEGNRVGVSIDGVNLPDSEENSLYARYGNFNSSRLSIDPELVRNIEIVKGADSFNTGSGALGGGVNYQTLQGRDLLLDDRQFGVMMKNGYSTRNREWTNTLGFGVSNDRVDAALLYSQRRGHETESAGNRGYAVEGEGSGANIRGSARGIPDSSKHKYNHHALGKIAYQINDNHRIGASLNGQQGHNYTVEESYNLTASSWREADDVNRRRNANLFYEWMPDSNWLSSLKADFDYQKTKVAAVNNKGSFPMDYSTWTRNYNQKDLDEIYNRSMDTRFKRFTLRLDSHPLQLGGGRHRLSFKTFVSRRDFENLNRDDYYFSGRVVRTTSSIQHPVKTTNYGFSLSDQIQWNDVFSSRAGIRYDHTKMTPQELNAECHACDKTPPAANTYKGWSGFVGLAAQLNQAWRVGYDITSGYRVPNASEVYFTYNHGSGNWLPNPNLKAERSTTHTLSLQGRSEKGMLDANLYQSNYRNFLSEEQKLTTSGTPGCTEENAYYGICSDPYKEKLDWQMKNIDKARIRGIELTGRLNVDKVASFVPEGWKLFGSLGYAKSKLSGDNSLLSTQPLKVIAGIDYESPSEKWGVFSRLTYLGAKKVKDAQYTVYENKGWGTPLQKKVKDYPWLNKSAYVFDMYGFYKPAKNLTLRAGVYNLFNRKYTTWDSLRGLYSYSTTNAVDRDGKGLDRYRAPGRNYAVSLEWKF

SEQ ID NO: 27 - protein, 591 aa, Neisseria meningitidis
MNKIYRIIWNSALNAWVVVSELTRNHTKRASATVKTAVLATLLFATVQASANNEEQEEDLYLDPVQRTVAVLIVNSDKEGTGEKEKVEENSDWAVYFNEKGVLTAREITLKAGDNLKIKQNGTNFTYSLKKDLTDLTSVGTEKLSFSANGNKVNITSDTKGLNFAKETAGTNGDTTVHLNGIGSTLTDTLLNTGATTNVTNDNVTDDEKKRAASVKDVLNAGWNIKGVKPGTTASDNVDFVRTYDTVEFLSADTKTTTVNVESKDNGKKTEVKIGAKTSVIKEKDGKLVTGKDKGENGSSTDEGEGLVTAKEVIDAVNKAGWRMKTTTANGQTGQADKFETVTSGTNVTFASGKGTTATVSKDDQGNITVMYDVNVGDALNVNQLQNSGWNLDSKAVAGSSGKVISGNVSPSKGKMDETVNINAGNNIEITRNGKNIDIATSMTPQFSSVSLGAGADAPTLSVDGDALNVGSKKDNKPVRITNVAPGVKEGDVTNVAQLKGVAQNLNNRIDNVDGNARAGIAQAIATAGLVQAYLPGKSMMAIGGGTYRGEAGYAIGYSSISDGGNWIIKGTASGNSRGHFGASASVGYQW

SEQ ID NO: 28 - protein, 1457 aa, Neisseria meningitidis
MKTTDKRTTETHRKAPKTGRIRFSPAYLAICLSFGILPQAWAGHTYFGINYQYYRDFAENKGKFAVGAKDIEVYNKKGELVGKSMTKAPMIDFSVVSRNGVAALVGDQYIVSVAHNGGYNNVDFGAEGRNPDQHRFTYKIVKRNNYKAGTKGHPYGGDYHMPRLHKFVTDAEPVEMTSYMDGRKYIDQNNYPDRVRIGAGRQYWRSDEDEPNNRESSYHIASAYSWLVGGNTFAQNGSGGGTVNLGSEKIKHSPYGFLPTGGSFGDSGSPMFIYDAQKQKWLINGVLQTGNPYIGKSNGFQLVRKDWFYDEIFAGDTHSVFYEPRQNGKYSFNDDNNGTGKINAKHEHNSLPNRLKTRTVQLFNVSLSETAREPVYHAAGGVNSYRPRLNNGENISFIDEGKGELILTSNINQGAGGLYFQGDFTVSPENNETWQGAGVHISEDSTVTWKVNGVANDRLSKIGKGTLHVQAKGENQGSISVGDGTVILDQQADDKGKKQAFSEIGLVSGRGTVQLNADNQFNPDKLYFGFRGGRLDLNGHSLSFHRIQNTDEGAMIVNHNQDKESTVTITGNKDIATTGNNNSLDSKKEIAYNGWFGEKDTTKTNGRLNLVYQPAAEDRTLLLSGGTNLNGNITQTNGKLFFSGRPTPHAYNHLNDHWSQKEGIPRGEIVWDNDWINRTFKAENFQIKGGQAVVSRNVAKVKGDWHLSNHAQAVFGVAPHQSHTICTRSDWTGLTNCVEKTITDDKVIASLTKTDISGNVDLADHAHLNLTGLATLNGNLSANGDTRYTVSHNATQNGNLSLVGNAQATFNQATLNGNTSASGNASFNLSDHAVQNGSLTLSGNAKANVSHSALNGNVSLADKAVFHFESSRFTGQISGGKDTALHLKDSEWTLPSGTELGNLNLDNATITLNSAYRHDAAGAQTGSATDAPRRRSRRSRRSLLSVTPPTSVESRFNTLTVNGKLNGQGTFRFMSELFGYRSDKLKLAESSEGTYTLAVNNTGNEPASLEQLTVVEGKDNKPLSENLNFTLQNEHVDAGAWRYQLIRKDGEFRLHNPVKEQELSDKLGKAEAKKQAEKDNAQSLDALIAAGRDAVEKTESVAEPARQAGGENVGIMQAEEEKKRVQADKDTALAKQREAETRPATTAFPRARRARRDLPQLQPQPQPQPQRDLISRYANSGLSEFSATLNSVFAVQDELDRVFAEDRRNAVWTSGIRDTKHYRSQDFRAYRQQTDLRQIGMQKNLGSGRVGILFSHNRTENTFDDGIGNSARLAHGAVFGQYGIDRFYIGISAGAGFSSGSLSDGIGGKIRRRVLHYGIQARYRAGFGGFGIEPHIGATRYFVQKADYRYENVNIATPGLAFNRYRAGIKADYSFKPAQHISITPYLSLSYTDAASGKVRTRVNTAVLAQDFGKTRSAEWGVNAEIKGFTLSLHAAAAKGPQLEAQHSAGIKLGYRW

SEQ ID NO: 29 - protein, 797 aa, Neisseria meningitidis
MKLKQIASALMMLGISPLALADFTIQDIRVEGLQRTEPSTVFNYLPVKVGDTYNDTHGSAIIKSLYATGFFDDVRVETADGQLLLTVIERPTIGSLNITGAKMLQNDAIKKNLESFGLAQSQYFNQATLNQAVAGLKEEYLGRGKLNIQITPKVTKLARNRVDIDITIDEGKSAKITDIEFEGNQVYSDRKLMRQMSLTEGGIWTWLTRSNQFNEQKFAQDMEKVTDFYQNNGYFDFRILDTDIQTNEDKTKQTIKITVHEGGRFRWGKVSIEGDTNEVPKAELEKLLTMKPGKWYERQQMTAVLGEIQNRMGSAGYAYSEISVQPLPNAETKTVDFVLHIEPGRKIYVNEIHITGNNKTRDEVVRRELRQMESAPYDTSKLQRSKERVELLGYFDNVQFDAVPLAGTPDKVDLNMSLTERSTGSLDLSAGWVQDTGLVMSAGVSQDNLFGTGKSAALRASRSKTTLNGSLSFTDPYFTADGVSLGYDVYGKAFDPRKASTSIKQYKTTTAGAGIRMSVPVTEYDRVNFGLVAEHLTVNTYNKAPKHYADFIKKYGKTDGTDGSFKGWLYKGTVGWGRNKTDSALWPTRGYLTGVNAEIALPGSKLQYYSATHNQTWFFPLSKTFTLMLGGEVGIAGGYGRTKEIPFFENFYGGGLGSVRGYESGTLGPKVYDEYGEKISYGGNKKANVSAELLFPMPGAKDARTVRLSLFADAGSVWDGKTYDDNSSSATGGRVQNIYGAGNTHKSTFTNELRYSAGGAVTWLSPLGPMKFSYAYPLKKKPEDEIQRFQFQLGTTF

SEQ ID NO: 30 - DNA, 14 bp, Artificial sequence
  FNR box from aniA
ttgacnnnnatcaa

SEQ ID NO: 31 - DNA, 6 bp, Artificial Sequence
  -10 hexamer
tataat

SEQ ID NO: 32 - DNA, 6 bp, Artificial Sequence
  -10 hexamer
ttgatc

(19) United States
(12) Patent Application Publication
(10) Pub. No.: US 2013/0022639 A1
(43) Pub. Date: Jan. 24, 2013

(54) EXPRESSION OF MENINGOCOCCAL FHBP POLYPEPTIDES

(71) Applicants: Francesca Oriente, Campobasso (IT); Isabel Delany, Siena (IT)

(72) Inventors: Francesca Oriente, Campobasso (IT); Isabel Delany, Siena (IT)

(73) Assignee: Novartis AG, Basel (CH)

(21) Appl. No.: 13/498,893

(22) PCT Filed: Sep. 30, 2010
(86) PCT No.: PCT/IB10/02557

§ 371 Date: Oct. 1, 2012

Related U.S. Application Data
(60) Provisional application No. 61/247,428, filed on Sep. 30, 2009.

(51) Int. Cl. A61K 39/095; A61P 31/04; C12N 15/31; C07K 14/22; C12N 1/21; C12N 15/74

(52) U.S. Cl. 424/250.1; 435/252.3; 435/471; 536/23.7; 435/320.1; 530/350

(57) ABSTRACT

The meningococcal fhbp gene (encoding factor H binding protein) is naturally expressed from two independent transcripts by two differentially regulated promoters. In one transcript it is co-expressed with the neighbouring upstream gene from the P_{nmb1869} promoter. The other transcript is monocistronic and is expressed from its own dedicated promoter, P_{fhbp}, which is activated by the global regulatory protein FNR in response to oxygen-limiting conditions. To increase expression of the monocistronic transcript a constitutively-active FNR mutant is used. The P_{fhbp} promoter can thus be activated, leading to over-expression of FNR-activated genes, such as fhbp.

This application claims the benefit of U.S. provisional application 61/247,428 filed Sep. 30, 2009, the complete contents of which are incorporated by reference herein.

### TECHNICAL FIELD

This invention is in the field of protein expression, and in particular expression of the Neisserial factor H binding protein (fHBP).

### BACKGROUND ART

Neisseria meningitidis is a Gram-negative encapsulated bacterial pathogen. One antigen of interest in developing a broad-spectrum vaccine against serogroup B meningococcus is fHBP, also known as protein ‘741’ [1], ‘NMB1870’, ‘GNA1870’ [2-4], ‘P2086’, ‘LP2086’ or ‘ORF2086’ [5-7]. This lipoprotein is expressed across all meningococcal serogroups and has been found in multiple strains. fHBP sequences have been grouped into three families [2] (referred to herein as families I, II & III), and serum raised against a given family is bactericidal within the same family, but is not active against strains which express one of the other families i.e. there is intra-family, but not inter-family, cross-protection.

For vaccination purposes, fHBP protein has been used as recombinant protein expressed in E. coli [8] or has been over-expressed in meningococcus such that outer membrane vesicles purified from the over-expressing strains will display large amounts of immunogenic fHBP [9]. These vesicles can then be used in vaccines to provide a strong and protective anti-fHBP response.

It is an object of the invention to provide further and improved approaches for increasing the expression of fHBP in meningococcus.

### DISCLOSURE OF THE INVENTION

The inventor has found that the fhbp gene is expressed from two independent transcripts by two differentially regulated promoters: In one transcript it is co-expressed with the neighbouring upstream gene (nmb1869) from the P_{nmb1869} promoter. The other transcript is monocistronic and is expressed from its own dedicated promoter, P_{fhbp}, which is activated by the global regulatory protein FNR (the known anaerobic activator protein, fumarate and nitrate reductase regulator) in response to oxygen limiting conditions. To increase expression of the monocistronic transcript a mutant form of FNR is used. The mutant form is constitutively active, even under aerobic conditions, and so the endogenous P_{fhbp} promoter is constitutively activated, leading to over-expression of fHBP. The same approach can be used to over-express any other meningococcal gene which has a FNR-activated promoter, including genes which are engineered to be under the control of such a promoter.

Thus the invention provides a meningococcus which (a) has a gene whose transcription is under the control of a FNR-activated promoter, and (b) expresses a constitutively active form of FNR. Expression of the FNR leads to constitutive expression of the FNR-activated gene. The gene whose transcription is under the control of a FNR-activated promoter is ideally a fhbp gene. Although not essential, the meningococcus ideally does not express a non-constitutively active form of FNR.

The invention also provides a process for preparing a mutant meningococcus, comprising a step of modifying its endogenous fnr gene such that the encoded FNR protein is constitutively active.

The invention also provides a process for preparing a mutant meningococcus, comprising a step of introducing a gene encoding a constitutively active form of FNR. This process may also include a step of modifying any endogenous fnr gene in the meningococcus to inhibit or prevent its expression.

Thus the invention also provides a process for increasing expression of a transcript whose transcription in a meningococcus is controlled by a FNR-activated promoter, comprising providing the meningococcus with a constitutively active form of FNR. Increased expression of the transcript can then provide increased levels of its encoded protein(s) in the meningococcus.

Thus the invention also provides a process for increasing expression of a transcript in a meningococcus, wherein the transcript contains a gene whose expression is controlled by at least two different promoters and wherein one of those two promoters is a FNR-activated promoter and the other is not, comprising providing the meningococcus with a constitutively active form of FNR, thereby increasing expression of the transcript. Of the multiple transcripts which include the gene, those driven by the FNR-activated promoter are increased relative to those driven by a different promoter.

The invention also provides a process for preparing a proteoliposomic meningococcal vesicle, comprising a step of treating a meningococcus of the invention to disrupt its outer membrane, thereby forming vesicles therefrom which include protein components of the outer membrane (such as fHBP). The vesicles can be used as immunogenic components in an immunogenic composition (e.g. as vaccine against meningococcus). The process may include a further step of separating the vesicles from any living and/or whole bacteria, such as by size separation (e.g. filtration, using a filter which allows the vesicles to pass through but which does not allow intact bacteria to pass through), or by centrifugation to preferentially pellet cells relative to the vesicles (e.g. low speed centrifugation).

The invention also provides a process for preparing an immunogenic composition (e.g. a vaccine) comprising a step of formulating vesicles prepared by the above process for preparing a vesicle with a pharmaceutically acceptable carrier (e.g. a buffer) and/or with an immunological adjuvant and/or with one or more further immunogenic components.

The invention also provides a meningococcus which expresses a constitutively active form of FNR.

The invention also provides a constitutively active form of meningococcus FNR, and in addition provides nucleic acid encoding such a FNR.

A particularly useful constitutively active form of FNR comprises SEQ ID NO: 5 which has, compared to the wild-type FNR sequence (SEQ ID NO: 4 from strain MC58), a mutation at Asp-148 e.g. D148A, in which wild-type Asp-148 is replaced with Ala.

The invention offers advantages when compared to existing strategies to over-express outer membrane proteins (e.g. fHBP) in meningococci. Although has been suggested to drive expression heterologous promoters to over-express proteins, these strategies result in over-expression only if the heterologous promoter has stronger basal activity than the endogenous promoter. The strategy described herein acts directly by increasing or enhancing the endogenous expression of a FNR-activated gene, which achieves over-expression without requiring promoter modification.

The Meningococcus

The invention provides various meningococci which express constitutively active FNR. Unlike normal wild-type strains, therefore, FNR-activated genes can be expressed at high levels even when oxygen levels are not limiting. Such genes include the fhbp genes, and so meningococci of the invention can over-express fHBP protein in their outer membranes.

Meningococci of the invention can be prepared from wild-type strains by directed mutagenesis, or by random mutagenesis followed by screening for the desired modifications, or by knock-out and knock-in techniques. For instance, the gene encoding an endogenous FNR can be modified using site-directed mutagenesis techniques to introduce a mutation which provides constitutive activity. In other embodiments, an endogenous fnr gene might be knocked out (e.g. by deletion, or by replacement with a marker) and a new fnr gene can be introduced (e.g. at the same site as the deletion, integrated on the chromosome but at a different site from the deletion, or on a plasmid). In other embodiments a new fnr gene is introduced while retaining the endogenous fnr gene. Various ways of achieving these and similar goals will be apparent. Integration between genes NMB1428 and NMB1429 is convenient.

Compared to normal wild-type strains, meningococci of the invention express constitutively active FNR. As well as this modification, meningococci may have at least one further modification when compared to wild-type strains e.g. introduced by genetic manipulation [10-13]. For instance, the meningococci may have been modified to increase immunogenicity (e.g. to hyper-express immunogens, including immunogens not activated by FNR), to reduce toxicity, to inhibit capsular polysaccharide synthesis, to down-regulate PorA expression, etc.

Meningococci of the invention may have a modified fur gene [14]. Reference 21 teaches that nspA expression should be up-regulated with concomitant porA and cps knockout, and these modifications may be used. The meningococci may express multiple different PorA subtypes [15]. Meningococci of the invention may have low endotoxin levels e.g. achieved by knockout of enzymes involved in LPS biosynthesis [16,17]. These or others mutants can all be used with the invention.

Meningococci of the invention may express more than one PorA subtype. 6-valent and 9-valent PorA strains have previously been constructed. The strain may express 2, 3, 4, 5, 6, 7, 8 or 9 of PorA subtypes: P1.7,16; P1.5-1,2-2; P1.19,15-1; P1.5-2,10; P1.12-1,13; P1.7-2,4; P1.22,14; P1.7-1,1 and/or P1.18-1,3,6. In other embodiments a strain may have been down-regulated for PorA expression e.g. in which the amount of PorA has been reduced by at least 20% (e.g. ≧30%, ≧40%, ≧50%, ≧60%, ≧70%, ≧80%, ≧90%, ≧95%, etc.), or even knocked out, relative to wild-type levels (e.g. relative to strain H44/76).

Meningococci of the invention may hyper-express (relative to the corresponding wild-type strain) certain proteins. For instance, strains may hyper-express NspA, protein 287 [18], TbpA and/or TbpB [19], Cu,Zn-superoxide dismutase [19], HmbR, etc.

In some embodiments a meningococcus may include one or more of the knockout and/or hyper-expression mutations disclosed in references 20 to 23. Preferred genes for down-regulation and/or knockout include: (a) Cps, CtrA, CtrB, CtrC, CtrD, FrpB, GalE, HtrB/MsbB, LbpA, LbpB, LpxK, Opa, Opc, PilC, PorB, SiaA, SiaB, SiaC, SiaD, TbpA, and/or TbpB [20]; (b) CtrA, CtrB, CtrC, CtrD, FrpB, GalE, HtrB/MsbB, LbpA, LbpB, LpxK, Opa, Opc, PhoP, PilC, PmrE, PmrF, SiaA, SiaB, SiaC, SiaD, TbpA, and/or TbpB [21]; (c) ExbB, ExbD, rmpM, CtrA, CtrB, CtrD, GalE, LbpA, LpbB, Opa, Opc, PilC, PorB, SiaA, SiaB, SiaC, SiaD, TbpA, and/or TbpB [22]; and (d) CtrA, CtrB, CtrD, FrpB, OpA, OpC, PilC, PorB, SiaD, SynA, SynB, and/or SynC [23].

Meningococci of the invention may have a MltA (NMB0033) knockout [24], which increases the strain's release of vesicles during normal growth. Such “hyperblebbing” strains are a useful source of immunogenic vesicles e.g. vesicles containing high levels of outer membrane proteins from over-expressed FNR-activated genes such as fhbp.

Meningococci of the invention may, in some embodiments, have one or more, or all, of the following characteristics: (i) down-regulated or knocked-out LgtB and/or GalE to truncate the meningococcal LOS; (ii) up-regulated TbpA; (iii) up-regulated NhhA; (iv) up-regulated Omp85; (v) up-regulated LbpA; (vi) up-regulated NspA; (vii) knocked-out PorA; (viii) down-regulated or knocked-out FrpB; (ix) down-regulated or knocked-out Opa; (x) down-regulated or knocked-out Opc; (xii) deleted cps gene complex. A truncated LOS can be one that does not include a sialyl-lacto-N-neotetraose epitope e.g. it might be a galactose-deficient LOS. The LOS may have no α chain.

Meningococci may contain genetic modifications which result in decreased or no detectable toxic activity of lipid A, particularly if they will be used for making proteoliposomic vesicles. Various modifications are known for reducing the toxic lipid A activity. For instance, the meningococcus may have a knockout of the lpxL1 and/or lpxL2 genes e.g. giving tetra- or penta-acylated lipid A. Mutations in the lipid A 4′-kinase gene (lpxK) also decreases the toxic activity of lipid A. LpxL1 knockout strains are preferred, particularly when fHBP expression is upregulated [25].

LPS toxic activity can also be altered by introducing mutations in genes/loci involved in polymyxin B resistance, such as pmrE and/or pmrF. Mutations in the PhoP-PhoQ regulatory system (a phospho-relay two component regulatory system) can also give a modified lipid A with reduced ability to stimulate E-selectin expression and TNF secretion.

Meningococci may contain more than one fhbp gene. For example, they may include a flzbp gene for more than one of the fHBP families I, II and III. For instance, reference 26 discloses a mutant strain with attenuated endotoxin that expresses both endogenous family I and heterologous family II variants. Vesicles prepared from such a strain offer a broader spectrum of anti-fHBP antibody responses. Each fhbp gene may be regulated by its own FNR-activated promoter, but it is also possible to include each fhbp gene in a polycistronic transcript (a single FNR regulon).

Meningococci of the invention may be modified to disrupt transcriptional termination from the P_{nmb1869} promoter.

Meningococci of the invention can be in any serogroup e.g. A, B, C, W135, Y. They will usually be serogroup B strains. The strain may be of any serotype (e.g. 1, 2a, 2b, 4, 14, 15, 16, etc.), any serosubtype, and any immunotype (e.g. L1; L2; L3; L3,3,7; L10; etc.). The meningococci may be from any suitable lineage, including hyperinvasive and hypervirulent lineages e.g. any of the following seven hypervirulent lineages: subgroup I; subgroup III; subgroup IV-1; ET-5 complex; ET-37 complex; A4 cluster; lineage 3.

Constitutively-Active FNR

FNR is a global anaerobic regulator which requires a [4Fe-4S] cluster for its activity under anaerobic conditions. The FNR polypeptide is synthesized during both aerobic and anaerobic growth, but the associated iron-sulfur center is degraded in aerobic cultures with a half life of about 2 minutes. The assembly of the [4Fe-4S] iron-sulfur center promotes dimerization during anaerobic growth, a prerequisite for FNR to bind to its inverted repeat target sequence at FNR-dependent promoters.

Meningococci of the invention express a constitutively-active FNR. It is known that FNR from E. coli can be modified such that its [4Fe-4S] cluster is O₂-stable, thereby giving a protein which is constitutively active i.e. it activates transcription of FNR-dependent genes even when oxygen is not limiting. Suitable mutations in the E. coli sequence include [27] modifications at Asp-22 (e.g. D22G), Leu-28 (e.g. L28H), His-93 (e.g. H93R), Glu-150 (e.g. E150K), and/or Asp-148 (e.g. D148A, D148G, D148V). The mutations may have various underlying functional effects e.g. to prevent change cAMP binding to FNR, to prevent oxidation of the [4Fe-4S] cluster, to promote dimerisation of FNR, etc. The published literature has already made analogous modifications to the gonococcal FNR (e.g. reference 28 confirms that the L28H and D148A mutants, which are at gonococcal residues 22 and 148, are active even in the presence of O₂) and the examples herein show that the meningococcal FNR can similarly be modified. An alignment of the E. coli and meningococcal FNR amino acid sequences (SEQ ID NOs: 4 and 6) is shown in the examples to aid in selecting further effective mutations of the meningococcal FNR.

Constitutively-active meningococcal FNRs of the invention can drive expression from FNR-activated meningococcal genes in an oxygen-dependent manner. Preferred constitutively-active FNRs are also resistant to inactivation by nitric oxide, which can nitrosylate the [4Fe-4S] cluster of the wild-type protein.

Methods for preparing mutant forms of wild-type meningococcal FNR proteins (e.g. mutants of SEQ ID NO: 4) are well known in the art e.g. by site-directed mutagenesis or error-prone PCR. Thus an endogenous fnr gene in a meningococcus, expressing an O₂-dependent FNR, can be modified such that the encoded FNR protein is instead constitutively active.

When a meningococcus of the invention expresses a constitutively-active FNR, it is preferred (but not necessary) that it does not also express a non-constitutively active form of FNR. Thus the constitutively-active FNR may be the only FNR which the meningococcus expresses. This can be achieved by modifying an endogenous fnr gene or, as an alternative, by inactivating an endogenous fnr gene and introducing a modified fnr gene encoding a constitutively active protein, or by introducing a fnr gene into the FNR null strain “MC-fnrKO” disclosed in reference 29.

The invention also provides a constitutively active meningococcal FNR. This can, compared to the wild-type FNR sequence (SEQ ID NO: 4 from strain MC58), have a mutation at one or more of residues Leu-22 (e.g. L22H), Glu-144 (e.g. E144K), and/or Asp-148 (e.g. D148A, D148G, D148V). For example, a constitutively active meningococcal FNR of the invention may comprise SEQ ID NO: 5, in which wild-type Asp-148 is replaced with Ala (i.e. the meningococcal mutation corresponding to the E. coli D148A mutant).

The invention also provides a transcription factor which can drive expression from a meningococcal FNR-activated promoter, wherein the factor comprises an amino acid sequence having at least x% sequence identity to SEQ ID NO: 4, provided that residue 148 of the amino acid sequence (numbered according to SEQ ID NO: 4) is not Asp (e.g. is Ala, Gly or Val).

The invention also provides nucleic acid encoding these FNR proteins. Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acids of the invention can take various forms e.g. single-stranded, double-stranded, vectors, primers, probes, labelled, unlabelled, etc.

Nucleic acids of the invention are preferably in isolated or substantially isolated form.

The term “nucleic acid” includes DNA and RNA, and also their analogues, such as those containing modified backbones, and also peptide nucleic acids (PNA), etc.

Nucleic acid according to the invention may be labelled e.g. with a radioactive or fluorescent label.

The invention also provides vectors (such as plasmids) comprising nucleotide sequences of the invention (e.g. cloning or expression vectors) and host cells transformed with such vectors.

FNR-Activated Genes and Promoters

Various genes in meningococci are transcribed from FNR-dependent promoters. For instance, reference 29 reports various FNR-dependent genes and operons which were identified by microarray experiments: 175 genes were differentially transcribed by more than 2-fold. FNR-activated genes include, but are not limited to, nmb1806, mapA, pgmβ, NMB0388, galM, nmb0363, nmb1805, nosR, nmb1677, aniA and fhbp. Increased expression of any of these genes (relative to wild-type) can be achieved, even in aerobic conditions, in a meningococcus which has a constitutively active FNR.

Any of these genes can be used as a source of a natural FNR-activated promoter, which may be linked to and thus drive expression from a downstream gene of interest. The invention can also be used with modified FNR-activated promoters. For instance, the examples show that the NM117 strain has a P_{fhbp} promoter with an inefficient −10 promoter element which does not exhibit over-expression by a constitutively active FNR. Thus a modified FNR-activated promoter useful with the invention may have a −10 and/or −35 hexamer which is the consensus for sigma 70 promoter (e.g. SEQ ID NO: 20 for −10, and SEQ ID NO: 21 for −35; or SEQ ID NO: 31 for −10, and SEQ ID NO: 32 for −35), and so it may be modified to bring its sequence closer (or completely) to the consensus. Similarly, a modified FNR-activated promoter useful with the invention may have a FNR-binding site (FNR-box) from a gene such as meningococcal aniA, for example SEQ ID NO: 30, which has a high affinity for FNR, or may have a modified FNR-binding site which brings its sequence closer (or completely) to the FNR-box consensus SEQ ID NO: 19. In general terms, therefore, a promoter may be constructed which is highly active when FNR is present e.g. by joining promoter elements (−10, −35 and FNR-box) from known FNR-activated promoters, including wild-type or optimised elements.

Although a meningococcus of the invention may have a constitutively active FNR, this constitutive activity is controlled at a post-translational level. To maximise the cytosolic levels of constitutively active FNR, therefore, the meningococcus should be grown under conditions where FNR is actively transcribed and translated.

The FNR-activated gene whose expression is achieved in meningococci of the invention can be an endogenous gene (e.g. an endogenous fhbp gene) under the control of an endogenous FNR-activated promoter, an endogenous gene under the control of an introduced FNR-activated promoter, an introduced gene under the control of an endogenous FNR-activated promoter, or an introduced gene under the control of an introduced FNR-activated promoter. Thus the invention may be used for over-expression of endogenous or exogenous proteins (e.g. as an alternative to the approaches given in reference 10), for instance by linking genes encoding outer membrane proteins to FNR-activated promoters, thereby increasing these proteins' levels in the outer membrane (and thus in vesicles).

The invention is particularly useful for expressing outer membrane proteins from FNR-dependent promoters. The protein, such as fHBP, can be over-expressed (relative to the wild-type strain) in the outer membrane and retained in proteoliposomic vesicles prepared from the meningococcus. The outer membrane protein can be in an immunoaccessible form in the vesicles i.e. an antibody which can bind to purified polypeptide of the invention can also bind to the polypeptide when present in the vesicles. The most preferred gene whose transcription is under the control of a FNR-activated promoter, and thus whose expression can be increased, is fhbp encoding the factor H binding protein.

Factor H Binding Protein

Full-length fHBP has amino acid sequence SEQ ID NO: 1 (strain MC58). The mature lipoprotein (N-terminal cysteine) lacks the first 19 amino acids of SEQ ID NO: 1, and the artificial ΔG form of fHBP lacks the first 26 amino acids. The MC58 sequence is in fHBP family I. Example sequences for families II and III are SEQ ID NO: 2 (family II; strain 2996) and SEQ ID NO: 3 (family III; strain M1239) and these are similarly lipidated at N-terminal cysteines in wild-type meningococci.

The promoter for the fhbp gene is activated by FNR and so the invention can be used to express any of these fHBP sequences in a meningococcus. More generally, the invention can be used to express a fhbp gene encoding an amino acid sequence comprising one of SEQ ID NOs: 1, 2, or 3, comprising (a) an amino acid sequence having at least x% sequence identity to any one of SEQ ID NOs: 1, 2 or 3, where the value of x is 65, 70, 75, 80, 85, 90, 95, 96, 97, 98, 99 or more; and/or (b) a fragment of at least n amino acids to any one of SEQ ID NOs: 1, 2 or 3, where the value of n is 7, 8, 9, 10, 11, 12, 13, 14, 15, 20, 25, 30, 35, 40, 45, 50, 60, 70, 80, 90, 100 or more. The fragment of (b) preferably comprises an epitope of the said SEQ ID NO. The protein encoded by the fhbp gene ideally has the ability, when administered to a host animal, to induce bactericidal anti-meningococcal antibodies. Further information on bactericidal responses is given below.

The fhbp gene, and/or its encoded amino acid sequence, may occur naturally or may be an artificial sequence. For instance, it is known to prepare artificial fHBP sequences which incorporate features from various different natural fHBP sequences e.g. see references 30 to 33. It is also known to create fusions of fHBP sequences from different families e.g. see references 33 to 36. The invention can be used with any of these artificial fHBP sequences. These methods can be used to provide fHBP proteins that can elicit antibodies which recognise more than one fHBP family. Thus the protein encoded by the fhbp gene may have the ability, when administered to a host animal, to induce bactericidal anti-meningococcal antibodies which recognise two or three of SEQ ID NOs 1, 2 and/or 3.

The fhbp gene might, for example, encode any of the following amino acid sequences: each of SEQ ID NOs: 1 to 45 of ref. 8; SEQ ID NOs: 79, 82, 83, 85, 87, 88, 89 and 90 of ref 8; SEQ ID NOs: 123 to 142 of ref. 8; each of the amino acid sequences within SEQ ID NOs: 1 to 329 of ref. 5; SEQ ID NOs: 2, 4, 6, 8, 10 or 12 of ref. 37; SEQ ID NOs: 43, 44, 52, 53, 62, 63, 64 or 65 of ref. 31; SEQ ID NOs: 46, 47, 48, 49, 50, 51, 52, 53, 54, 55, 56, 58, 59, 60, 63, 64, 65, 86, 87, 88, 89, 90, 91, 92, 93, 94 or 95 of ref. 32; each of SEQ ID NOs: 4 to 80 from ref. 30; each of SEQ ID NOs: 4 to 78 of ref. 38; each of SEQ ID NOs: 103 to 138 of ref. 38. For instance, the fhbp gene might encode an amino acid sequence comprising any of SEQ ID NOs: 12, 13 and 14 herein (known as 9C, 10A and 8B).

Proteoliposomic Vesicles

Meningococci of the invention are particularly useful for preparing proteoliposomic vesicles which retain outer membrane proteins from the bacterium. Over-expression of fHBP by using a constitutively active FNR, for instance, can be used to provide vesicles which are enriched for fHBP.

These proteoliposomic vesicle can be obtained by disruption of or blebbling from the outer membrane to form vesicles therefrom that include protein components of the outer membrane. Thus the term includes OMVs, blebs, microvesicles (MVs [39]) and ‘native OMVs’ (‘NOMVs’ [40]).

Blebs, MVs and NOMVs are naturally-occurring membrane vesicles that form spontaneously during bacterial growth and are released into culture medium. MVs can be obtained by culturing Neisseria in broth culture medium, separating whole cells from the smaller MVs in the broth culture medium (e,g. by filtration or by low-speed centrifugation to pellet only the cells and not the smaller vesicles), and then collecting the MVs from the cell-depleted medium (e.g. by filtration, by differential precipitation or aggregation of MVs, by high-speed centrifugation to pellet the MVs). Strains for use in production of MVs can generally be selected on the basis of the amount of MVs produced in culture e.g. refs. 41 & 42 describe Neisseria with high MV production. Hyperblebbing strains are disclosed in reference 43. Disruption of the mltA gene [24] can also provide strains which spontaneously release suitable vesicles during culture.

OMVs are prepared artificially from bacteria, and may be prepared using detergent treatment (e.g. with deoxycholate), or by non-detergent means (e.g. see reference 44). Techniques for forming OMVs include treating bacteria with a bile acid salt detergent (e.g. salts of lithocholic acid, chenodeoxycholic acid, ursodeoxycholic acid, deoxycholic acid, cholic acid, ursocholic acid, etc., with sodium deoxycholate [45 & 46] being preferred for treating Neisseria) at a pH sufficiently high not to precipitate the detergent [47]. Other techniques may be performed substantially in the absence of detergent [44] using techniques such as sonication, homogenisation, microfluidisation, cavitation, osmotic shock, grinding, French press, blending, etc. Methods using no or low detergent can retain useful antigens such as NspA [44]. Thus a method may use an OMV extraction buffer with about 0.5% deoxycholate or lower e.g. about 0.2%, about 0.1%, <0.05% or zero.

A useful process for OMV preparation is described in reference 48 and involves ultrafiltration on crude OMVs, rather than instead of high speed centrifugation. The process may involve a step of ultracentrifugation after the ultrafiltration takes place.

If LOS is present in a vesicle it is possible to treat the vesicle so as to link its LOS and protein components (“intra-bleb” conjugation [23]).

The proteoliposomic vesicles can be used as immunogenic components in an immunogenic composition. A process in which vesicles are formed may include a further step of separating the vesicles from any living and/or whole bacteria, such as by size separation (e.g. filtration, using a filter which allows the vesicles to pass through but which does not allow intact bacteria to pass through), or by centrifugation to preferentially pellet cells relative to the vesicles (e.g. low speed centrifugation).

Immunogenic Compositions

The invention provides immunogenic compositions comprising proteoliposomic vesicles of the invention. These compositions can be prepared by formulating the vesicles with a pharmaceutically acceptable carrier and/or with an immunological adjuvant and/or with one or more further immunogenic components.

The immunogenic composition may include a pharmaceutically acceptable carrier, which can be any substance that does not itself induce the production of antibodies harmful to the patient receiving the composition, and which can be administered without undue toxicity. Pharmaceutically acceptable carriers can include liquids such as water, saline, glycerol and ethanol. Auxiliary substances, such as wetting or emulsifying agents, pH buffering substances, and the like, can also be present in such vehicles. A thorough discussion of suitable carriers is available in ref. 49.

Neisserial infections affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared. The composition may be prepared for topical administration e.g. as an ointment, cream or powder. The composition be prepared for oral administration e.g. as a tablet or capsule, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops.

The composition is preferably sterile. It is preferably pyrogen-free. It is preferably buffered e.g. at between pH 6 and pH 8, generally around pH 7. Where a composition comprises an aluminium hydroxide salt, it is useful to include a histidine buffer [50]. Compositions of the invention may be isotonic with respect to humans.

Immunogenic compositions comprise an immunologically effective amount of immunogen, as well as any other of other specified components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

Previous work with meningococcal vesicle vaccines offers pharmaceutical, posological and formulation guidance for performing the invention. For example, VA-MENGOC-BC™ is an injectable suspension in 0.5 ml that contains 50 μg OMV from strain Cu-385-83 and 50 μg serogroup C capsular polysaccharide, absorbed to 2 mg of an aluminium hydroxide gel, plus 0.01% thiomersal and phosphate buffer. McNZB™ is also a 0.5 ml suspension, and contains 25 μg OMV from strain NZ98/254 adsorbed on 1.65 mg of an aluminium hydroxide adjuvant, with a histidine buffer and sodium chloride. MenBvac is similar to MeNZB™, but is prepared from strain 44/76. The concentration of OMVs for each subtype will be high enough to provide protective immunity after administration to a patient, either by a single dose schedule or a multiple dose schedule (e.g. including booster doses). The concentration of OMVs in compositions of the invention will generally be between 10 and 500 μg/ml, preferably between 25 and 200 μg/ml, and more preferably about 50 μg/ml or about 100 μg/ml (expressed in terms of total protein in the OMVs).

The composition may be administered in conjunction with other immunoregulatory agents.

Adjuvants which may be used in compositions of the invention include, but are not limited to:

A. Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc. [e.g. see chapters 8 & 9 of ref. 54], or mixtures of different mineral compounds, with the compounds taking any suitable form (e.g. gel, crystalline, amorphous, etc.), and with adsorption being preferred. The mineral containing compositions may also be formulated as a particle of metal salt.

The adjuvants known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ [chapter 9 of ref. 54]. The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

The adjuvants known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 1.2. Hydroxyphosphates can be distinguished from strict AlPO₄ by the presence of hydroxyl groups. For example, an IR spectrum band at 3164 cm⁻¹ (e.g. at 200° C.) indicates the presence of structural hydroxyls [ch. 9 of ref 54].

The PO₄/Al³⁺ molar ratio of an aluminium phosphate adjuvant will generally be between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs). Typical diameters of the particles are in the range 0.5-20 μm (e.g. about 5-10 m) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

Suspensions of aluminium salts used to prepare compositions of the invention may contain a buffer (e.g. a phosphate or a histidine or a Tris buffer), but this is not always necessary. The suspensions are preferably sterile and pyrogen-free. A suspension may include free aqueous phosphate ions e.g. present at a concentration between 1.0 and 20 mM, preferably between 5 and 15 mM, and more preferably about 10 mM. The suspensions may also comprise sodium chloride.

In one embodiment, an adjuvant component includes a mixture of both an aluminium hydroxide and an aluminium phosphate. In this case there may be more aluminium phosphate than hydroxide e.g. a weight ratio of at least 2:1 e.g. ≧5:1, ≧6:1, ≧7:1, ≧8:1, ≧9:1, etc.

The concentration of Al⁺⁺⁺ in a composition for administration to a patient is preferably less than 10 mg/ml e.g. ≦5 mg/ml, ≦4 mg/ml, ≦3 mg/ml, ≦2 mg/ml, ≦1 mg/ml, etc. A preferred range is between 0.3 and 1 mg/ml. A maximum of <0.85 mg/dose is preferred.

B. Oil Emulsions

Oil emulsion compositions suitable for use as adjuvants in the invention include squalene-water emulsions, such as MF59 [Chapter 10 of ref. 54; see also ref. 51] (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

Various suitable oin-in-water emulsions are known, and they typically include at least one oil and at least one surfactant, with the oil(s) and surfactant(s) being biodegradable (metabolisable) and biocompatible. The oil droplets in the emulsion are generally less than 5 μm in diameter, and advantageously the emulsion comprises oil droplets with a sub-micron diameter, with these small sizes being achieved with a microfluidiser to provide stable emulsions. Droplets with a size less than 220 nm are preferred as they can be subjected to filter sterilization.

The invention can be used with oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoid known as squalene, 2,6,10,15,19,23-hexamethyl-2,6,10,14,18,22-tetracosahexaene. Other preferred oils are the tocopherols (see below). Oil in water emulsions comprising sqlauene are particularly preferred. Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-6301NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Preferred surfactants for including in the emulsion are Tween 80 (polyoxyethylene sorbitan monooleate), Span 85 (sorbitan trioleate), lecithin and Triton X-100. As mentioned above, detergents such as Tween 80 may contribute to the thermal stability seen in the examples below.

Mixtures of surfactants can be used e.g. Tween 80/Span 85 mixtures. A combination of a polyoxyethylene sorbitan ester such as polyoxyethylene sorbitan monooleate (Tween 80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Specific oil-in-water emulsion adjuvants useful with the invention include, but are not limited to:
A submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [51-53], as described in more detail in Chapter 10 of ref. 54 and chapter 12 of ref 55. The MF59 emulsion advantageously includes citrate ions e.g. 10 mM sodium citrate buffer.
An emulsion comprising squalene, an a-tocopherol, and polysorbate 80. These emulsions may have from 2 to 10% squalene, from 2 to 10% tocopherol and from 0.3 to 3% Tween 80, and the weight ratio of squalene:tocopherol is preferably ≦1 (e.g. 0.90) as this provides a more stable emulsion. Squalene and Tween 80 may be present volume ratio of about 5:2, or at a weight ratio of about 11:5. One such emulsion can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, preferably about 180 nm.
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g. polysorbate 80), a Triton detergent (e.g. Triton X-100) and a tocopherol (e.g. an α-tocopherol succinate). The emulsion may include these three components at a mass ratio of about 75:11:10 (e.g. 750 μg/ml polysorbate 80, 110 μg/ml Triton X-100 and 100 μg/ml α-tocopherol succinate), and these concentrations should include any contribution of these components from antigens. The emulsion may also include squalene. The emulsion may also include a 3d-MPL (see below). The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [56] (0.05-1% Thr-MDP, 5% squalane, 2.5% Pluronic L121 and 0.2% polysorbate 80). It can also be used without the Thr-MDP, as in the “AF” adjuvant [57] (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm [58]. The emulsion may also include one or more of: alditol; a cryoprotective agent (e.g. a sugar, such as dodecylmaltoside and/or sucrose); and/or an alkylpolyglycoside. Such emulsions may be lyophilized.
An emulsion having from 0.5-50% of an oil, 0.1-10% of a phospholipid, and 0.05-5% of a non-ionic surfactant. As described in reference 59, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 60, produced by addition of aliphatic amine to desacylsaponin via the carboxyl group of glucuronic acid), dimethyidioctadecylammonium bromide and/or N,N-dioctadecyl-N,N-bis (2-hydroxyethyl)propanediamine.
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) [61].
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) [61].
An emulsion in which a saponin (e.g. QuilA or QS21) and a sterol (e.g. a cholesterol) are associated as helical micelles [62].

Antigens and adjuvants in a composition will typically be in admixture at the time of delivery to a patient. The emulsions may be mixed with antigen during manufacture, or extemporaneously, at the time of delivery. Thus the adjuvant and antigen may be kept separately in a packaged or distributed vaccine, ready for final formulation at the time of use. The antigen will generally be in an aqueous form, such that the vaccine is finally prepared by mixing two liquids. The volume ratio of the two liquids for mixing can vary (e.g. between 5:1 and 1:5) but is generally about 1:1.

C. Saponin Formulations [Chapter 22 of ref 54]

Saponin formulations may also be used as adjuvants in the invention. Saponins are a heterogeneous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 63. Saponin formulations may also comprise a sterol, such as cholesterol [64].

Combinations of saponins and cholesterols can be used to form unique particles called immunostimulating complexs (ISCOMs; see chapter 23 of ref. 54; also refs 65 & 66). ISCOMs typically also include a phospholipid such as phosphatidylethanolamine or phosphatidylcholine. Any known saponin can be used in ISCOMs. Preferably, the ISCOM includes one or more of QuilA, QHA & QHC. Optionally, the ISCOMS may be devoid of additional detergent [67].

A review of the development of saponin based adjuvants can be found in refs. 68 & 69.

D. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 70. Such “small particles” of 3dMPL are small enough to be sterile filtered through a 0.22μm membrane [70]. Other non-toxic LPS derivatives include monophosphoryl lipid A mimics, such as aminoalkyl glucosaminide phosphate derivatives e.g. RC-529 [71,72].

Lipid A derivatives include derivatives of lipid A from Escherichia coli such as OM-174. OM-174 is described for example in refs. 73 & 74.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 75, 76 and 77 disclose possible analog substitutions e.g. replacement of guanosine with 2′-deoxy-7-deazaguanosine. The adjuvant effect of CpG oligonucleotides is further discussed in refs. 78-83.

The CpG sequence may be directed to TLR9, such as the motif GTCGTT or TTCGTT [84]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 85-87. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 88-90.

A particularly useful adjuvant based around immunostimulatory oligonucleotides is known as IC-31™ [91-93]. Thus an adjuvant used with the invention may comprise a mixture of (i) an oligonucleotide (e.g. between 15-40 nucleotides) including at least one (and preferably multiple) CpI motifs (i.e. a cytosine linked to an inosine to form a dinucleotide), and (ii) a polycationic polymer, such as an oligopeptide (e.g. between 5-20 amino acids) including at least one (and preferably multiple) Lys-Arg-Lys tripeptide sequence(s). The oligonucleotide may be a deoxynucleotide comprising 26-mer sequence 5′-(IC)₁₃-3′ (SEQ ID NO: 7). The polycationic polymer may be a peptide comprising 11-mer amino acid sequence KLKLLLLLKLK (SEQ ID NO: 8). This combination of SEQ ID NOs: 7 and 8 provides the IC-31™ adjuvant.

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref. 94 and as parenteral adjuvants in ref. 95. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G 192. The use of ADP-ribosylating toxins and detoxified derivatives thereof, particularly LT-K63 and LT-R72, as adjuvants can be found in refs. 96-103. A useful CT mutant is or CT-E29H [104]. Numerical reference for amino acid substitutions is preferably based on the alignments of the A and B subunits of ADP-ribosylating toxins set forth in ref. 105, specifically incorporated herein by reference in its entirety.

E. Human Immunomodulators

Human immunomodulators suitable for use as adjuvants in the invention include cytokines, such as interleukins (e.g. IL-1, IL-2, IL-4, IL-5, IL-6, IL-7, IL-12 [106], etc.) [107], interferons (e.g. interferon-γ), macrophage colony stimulating factor, and tumor necrosis factor. A preferred immunomodulator is IL-12.

F. Bioadhesives and Mucoadhesives

Bioadhesives and mucoadhesives may also be used as adjuvants in the invention. Suitable bioadhesives include esterified hyaluronic acid microspheres [108] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [109].

G. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

H. Liposomes (Chapters 13 & 14 of ref 54)

Examples of liposome formulations suitable for use as adjuvants are described in refs. 110-112.

I. Imidazoquinolone Compounds.

Examples of imidazoquinolone compounds suitable for use adjuvants in the invention include Imiquamod and its homologues (e.g. “Resiquimod 3M”), described further in refs. 113 and 114.

The invention may also comprise combinations of aspects of one or more of the adjuvants identified above. For example, the following adjuvant compositions may be used in the invention: (1) a saponin and an oil-in-water emulsion [115]; (2) a saponin (e.g. QS21)+a non-toxic LPS derivative (e.g. 3dMPL) [116]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [117]; (5) combinations of 3dMPL with, for example, QS21 and/or oil-in-water emulsions [118]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3dMPL).

Other substances that act as immunostimulating agents are disclosed in chapter 7 of ref. 54.

An aluminium hydroxide adjuvant is useful, and antigens are generally adsorbed to this salt. Oil-in-water emulsions comprising squalene, with submicron oil droplets, are also preferred, particularly in the elderly. Useful adjuvant combinations include combinations of Th1 and Th2 adjuvants such as CpG & an aluminium salt, or resiquimod & an aluminium salt. A combination of an aluminium salt and 3dMPL may be used.

In addition to the vesicles, an immunogenic composition may include one or more further immunogenic components. Such components include, but are not limited to, further meningococcal antigen(s) and/or non-meningococcal antigen(s).

Meningococcal Antigens

As well as including a vesicle as described above, a composition of the invention may also include one or more further meningococcal antigen(s) to increase the breadth of strain coverage. Thus a composition can include a polypeptide or saccharide that, when administered to a mammal, elicits an antibody response that is bactericidal against meningococcus.

A composition may include a purified meningococcal antigen. Further details of meningococcal antigens are given below. For instance, it might include meningococcal antigen 287, NadA, NspA, HmbR, NhhA, App, 936, Omp85 or extra fHBP. A composition (see refs. 119 & 120) may include one or more of: a polypeptide comprising SEQ ID NO: 9; a polypeptide comprising SEQ ID NO: 10; and/or a polypeptide comprising SEQ ID NO: 11 (or a polypeptide comprising amino acids 24-350 of SEQ ID NO: 11). These polypeptides are preferably expressed recombinantly in a heterologous host and then purified e.g. for mixing with the vesicles. A composition may include a meningococcal capsular saccharide antigen e.g. as a conjugate.

A composition of the invention may include a 287 antigen. The 287 antigen was included in the published genome sequence for meningococcal serogroup B strain MC58 [121] as gene NMB2132 (GenBank accession number GI:7227388; SEQ ID NO: 23 herein). The sequences of 287 antigen from many strains have been published since then. For example, allelic forms of 287 can be seen in FIGS. 5 and 15 of reference 122, and in example 13 and FIG. 21 of reference 123 (SEQ IDs 3179 to 3184 therein). Various immunogenic fragments of the 287 antigen have also been reported. Preferred 287 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 23; and/or (b) comprising a fragment of at least ‘n’ consecutive amino acids of SEQ ID NO: 23, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 23. The most useful 287 antigens of the invention can elicit antibodies which, after administration to a subject, can bind to a meningococcal polypeptide consisting of amino acid sequence SEQ ID NO: 23. Advantageous 287 antigens for use with the invention can elicit bactericidal anti-meningococcal antibodies after administration to a subject.

A composition of the invention may include a NadA antigen. The NadA antigen was included in the published genome sequence for meningococcal serogroup B strain MC58 [121] as gene NMB1994 (GenBank accession number GI:7227256; SEQ ID NO: 24 herein). The sequences of NadA antigen from many strains have been published since then, and the protein's activity as a Neisserial adhesin has been well documented. Various immunogenic fragments of NadA have also been reported. Preferred NadA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 24; and/or (b) comprising a fragment of at least ‘n’ consecutive amino acids of SEQ ID NO: 24, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 24. The most useful NadA antigens of the invention can elicit antibodies which, after administration to a subject, can bind to a meningococcal polypeptide consisting of amino acid sequence SEQ ID NO: 24. Advantageous NadA antigens for use with the invention can elicit bactericidal anti-meningococcal antibodies after administration to a subject. One such fragment is amino acids 24-350 of SEQ ID NO: 11.

A composition of the invention may include a NspA antigen. The NspA antigen was included in the published genome sequence for meningococcal serogroup B strain MC58 [121] as gene NMB0663 (GenBank accession number GI:7225888; SEQ ID NO: 25 herein). The antigen was previously known from references 124 & 125. The sequences of NspA antigen from many strains have been published since then. Various immunogenic fragments of NspA have also been reported. Preferred NspA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 25; and/or (b) comprising a fragment of at least ‘n’ consecutive amino acids of SEQ ID NO: 25, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 25. The most useful NspA antigens of the invention can elicit antibodies which, after administration to a subject, can bind to a meningococcal polypeptide consisting of amino acid sequence SEQ ID NO: 25. Advantageous NspA antigens for use with the invention can elicit bactericidal anti-meningococcal antibodies after administration to a subject.

Compositions of the invention may include a meningococcal HmbR antigen. The full-length HmbR sequence was included in the published genome sequence for meningococcal serogroup B strain MC58 [121] as gene NMB1668 (SEQ ID NO: 26 herein). The invention can use a polypeptide that comprises a full-length HmbR sequence, but it will often use a polypeptide that comprises a partial HmbR sequence. Thus in some embodiments a HmbR sequence used according to the invention may comprise an amino acid sequence having at least i% sequence identity to SEQ ID NO: 26, where the value of i is 50, 60, 70, 80, 90, 95, 99 or more. In other embodiments a HmbR sequence used according to the invention may comprise a fragment of at least j consecutive amino acids from SEQ ID NO: 26, where the value of j is 7, 8, 10, 12, 14, 16, 18, 20, 25, 30, 35, 40, 50, 60, 70, 80, 90, 100, 150, 200, 250 or more. In other embodiments a HmbR sequence used according to the invention may comprise an amino acid sequence (i) having at least i% sequence identity to SEQ ID NO: 26 and/or (ii) comprising a fragment of at least j consecutive amino acids from SEQ ID NO: 26. Preferred fragments of j amino acids comprise an epitope from SEQ ID NO: 26. Such epitopes will usually comprise amino acids that are located on the surface of HmbR. Useful epitopes include those with amino acids involved in HmbR's binding to haemoglobin, as antibodies that bind to these epitopes can block the ability of a bacterium to bind to host haemoglobin. The topology of HmbR, and its critical functional residues, were investigated in reference 126. The most useful HmbR antigens of the invention can elicit antibodies which, after administration to a subject, can bind to a meningococcal polypeptide consisting of amino acid sequence SEQ ID NO: 26. Advantageous HmbR antigens for use with the invention can elicit bactericidal anti-meningococcal antibodies after administration to a subject.

A composition of the invention may include a NhhA antigen. The NhhA antigen was included in the published genome sequence for meningococcal serogroup B strain MC58 [121] as gene NMB0992 (GenBank accession number GI:7226232; SEQ ID NO: 27 herein). The sequences of NhhA antigen from many strains have been published since e.g. refs 122 & 127, and various immunogenic fragments of NhhA have been reported. It is also known as Hsf. Preferred NhhA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 27; and/or (b) comprising a fragment of at least ‘n’ consecutive amino acids of SEQ ID NO: 27, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 27. The most useful NhhA antigens of the invention can elicit antibodies which, after administration to a subject, can bind to a meningococcal polypeptide consisting of amino acid sequence SEQ ID NO: 27. Advantageous NhhA antigens for use with the invention can elicit bactericidal anti-meningococcal antibodies after administration to a subject.

A composition of the invention may include an App antigen. The App antigen was included in the published genome sequence for meningococcal serogroup B strain MC58 [121] as gene NMB1985 (GenBank accession number GI:7227246; SEQ ID NO: 28 herein). The sequences of App antigen from many strains have been published since then. Various immunogenic fragments of App have also been reported. Preferred App antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 28; and/or (b) comprising a fragment of at least ‘n’ consecutive amino acids of SEQ ID NO: 28, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 28. The most useful App antigens of the invention can elicit antibodies which, after administration to a subject, can bind to a meningococcal polypeptide consisting of amino acid sequence SEQ ID NO: 28. Advantageous App antigens for use with the invention can elicit bactericidal anti-meningococcal antibodies after administration to a subject.

A composition of the invention may include an Omp85 antigen. The Omp85 antigen was included in the published genome sequence for meningococcal serogroup B strain MC58 [121] as gene NMB0182 (GenBank accession number GI:7225401; SEQ ID NO: 29 herein). The sequences of Omp85 antigen from many strains have been published since then. Further information on Omp85 can be found in references 128 and 129. Various immunogenic fragments of Omp85 have also been reported. Preferred Omp85 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 29; and/or (b) comprising a fragment of at least ‘n’ consecutive amino acids of SEQ ID NO: 29, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 29. The most useful Omp85 antigens of the invention can elicit antibodies which, after administration to a subject, can bind to a meningococcal polypeptide consisting of amino acid sequence SEQ ID NO: 29. Advantageous Omp85 antigens for use with the invention can elicit bactericidal anti-meningococcal antibodies after administration to a subject.

A composition of the invention may include a 936 antigen. The 936 antigen was included in the published genome sequence for meningococcal serogroup B strain MC58 [130] as gene NMB2091 (SEQ ID NO: 22 herein). Preferred 936 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 22; and/or (b) comprising a fragment of at least ‘n’ consecutive amino acids of SEQ ID NO: 22, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 22. The most useful 936 antigens of the invention can elicit antibodies which, after administration to a subject, can bind to a meningococcal polypeptide consisting of amino acid sequence SEQ ID NO: 22. The 936 antigen is a good fusion partner for fHBP (e.g. see references 119 & 120).

These antigens are preferably prepared in substantially pure or substantially isolated form (i.e. substantially free from other Neisserial or host cell polypeptides) or substantially isolated form. In general, the polypeptides are provided in a non-naturally occurring environment e.g. they are separated from their naturally-occurring environment. In certain embodiments, the subject polypeptide is present in a composition that is enriched for the polypeptide as compared to a control. As such, purified polypeptide is provided, whereby purified is meant that the polypeptide is present in a composition that is substantially free of other expressed polypeptides, where by substantially free is meant that less than 90%, usually less than 60% and more usually less than 50% of the composition is made up of other expressed polypeptides.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains.

A composition of the invention can include conjugated capsular saccharide antigens from 1, 2, 3 or 4 of meningococcus serogroups A, C, W135 and Y.

Current serogroup C vaccines (Menjugate™ [131], Meningitec™ and NeisVac-C™) include conjugated saccharides. Menjugate™ and Meningitec™ have oligosaccharide antigens conjugated to a CRM₁₉₇ carrier, whereas NeisVac-C™ uses the complete polysaccharide (de-O-acetylated) conjugated to a tetanus toxoid carrier. The Menactra™ vaccine contains conjugated capsular saccharide antigens from each of serogroups Y, W135, C and A.

Compositions of the present invention may include capsular saccharide antigens from one or more of meningococcus serogroups Y, W135, C and A, wherein the antigens are conjugated to carrier protein(s) and/or are oligosaccharides. For example, the composition may include a capsular saccharide antigen from: serogroup C; serogroups A and C; serogroups A, C and W135; serogroups A, C and Y; serogroups C, W135 and Y; or from all four of serogroups A, C, W135 and Y.

A typical quantity of each meningococcal saccharide antigen per dose is between 1 μg and 20 μg e.g. about about 2.5 μg, about 4 μg, about 5 μg, or about 10 μg (expressed as saccharide).

Where a mixture comprises capsular saccharides from both serogroups A and C, the ratio (w/w) of MenA saccharide:MenC saccharide may be greater than 1 (e.g. 2:1, 3:1, 4:1, 5:1, 10:1 or higher). Where a mixture comprises capsular saccharides from serogroup Y and one or both of serogroups C and W135, the ratio (w/w) of MenY saccharide:MenW135 saccharide may be greater than 1 (e.g. 2:1, 3:1, 4:1, 5:1, 10:1 or higher) and/or that the ratio (w/w) of MenY saccharide:MenC saccharide may be less than 1 (e.g. 1:2, 1:3, 1:4, 1:5, or lower). Preferred ratios (w/w) for saccharides from serogroups A:C:W135:Y are: 1:1:1:1; 1:1:1:2; 2:1:1:1; 4:2:1:1; 8:4:2:1; 4:2:1:2; 8:4:1:2; 4:2:2:1; 2:2:1:1; 4:4:2:1; 2:2:1:2; 4:4:1:2; and 2:2:2:1. Preferred ratios (w/w) for saccharides from serogroups C:W135:Y are: 1:1:1; 1:1:2; 1:1:1; 2:1:1; 4:2:1; 2:1:2; 4:1:2; 2:2:1; and 2:1:1. Using a substantially equal mass of each saccharide is preferred.

Capsular saccharides may be used in the form of oligosaccharides. These are conveniently formed by fragmentation of purified capsular polysaccharide (e.g. by hydrolysis), which will usually be followed by purification of the fragments of the desired size.

Fragmentation of polysaccharides can be performed to give a final average degree of polymerisation (DP) in the oligosaccharide of less than 30 (e.g. between 10 and 20, preferably around 10 for serogroup A; between 15 and 25 for serogroups W135 and Y, preferably around 15-20; between 12 and 22 for serogroup C; etc.). DP can conveniently be measured by ion exchange chromatography or by colorimetric assays [132].

If hydrolysis is performed, the hydrolysate will generally be sized in order to remove short-length oligosaccharides. This can be achieved in various ways, such as ultrafiltration followed by ion-exchange chromatography. Oligosaccharides with a degree of polymerisation of less than or equal to about 6 are preferably removed for serogroup A, and those less than around 4 are preferably removed for serogroups W135 and Y.

Preferred MenC saccharide antigens are disclosed in reference 131, as used in Menjugate™.

The saccharide antigen may be chemically modified. This is particularly useful for reducing hydrolysis for serogroup A [133; see below]. De-O-acetylation of meningococcal saccharides can be performed. For oligosaccharides, modification may take place before or after depolymerisation.

Where a composition of the invention includes a MenA saccharide antigen, the antigen is preferably a modified saccharide in which one or more of the hydroxyl groups on the native saccharide has/have been replaced by a blocking group [133]. This modification improves resistance to hydrolysis.

Capsular saccharides in compositions of the invention will usually be conjugated to carrier protein(s). In general, conjugation enhances the immunogenicity of saccharides as it converts them from T-independent antigens to T-dependent antigens, thus allowing priming for immunological memory. Conjugation is particularly useful for paediatric vaccines and is a well known technique.

Typical carrier proteins are bacterial toxins, such as diphtheria or tetanus toxins, or toxoids or mutants thereof. The CRM₁₉₇ diphtheria toxin mutant [134] is useful, and is the carrier in the PREVNAR™ product. Other suitable carrier proteins include the N. meningitidis outer membrane protein complex [135], synthetic peptides [136,137], heat shock proteins [138,139], pertussis proteins [140,141], cytokines [142], lymphokines [142], hormones [142], growth factors [142], artificial proteins comprising multiple human CD4⁺ T cell epitopes from various pathogen-derived antigens such as N19 [144], protein D from H. influenzae [145-147], pneumolysin [148] or its non-toxic derivatives [149], pneumococcal surface protein PspA [150], iron-uptake proteins [151], toxin A or B from C. difficile [152], recombinant P. aeruginosa exoprotein A (rEPA) [153], etc.

Any suitable conjugation reaction can be used, with any suitable linker where necessary.

The saccharide will typically be activated or functionalised prior to conjugation. Activation may involve, for example, cyanylating reagents such as CDAP (e.g. 1-cyano-4-dimethylamino pyridinium tetrafluoroborate [154,155,etc.]). Other suitable techniques use carbodiimides, hydrazides, active esters, norborane, p-nitrobenzoic acid, N-hydroxysuccinimide, S-NHS, EDC, TSTU, etc.

Linkages via a linker group may be made using any known procedure, for example, the procedures described in references 156 and 157. One type of linkage involves reductive amination of the polysaccharide, coupling the resulting amino group with one end of an adipic acid linker group, and then coupling a protein to the other end of the adipic acid linker group [158,159]. Other linkers include B-propionamido [160], nitrophenyl-ethylamine [161], haloacyl halides [162], glycosidic linkages [163], 6-aminocaproic acid [164], ADH [165], C₄ to C₁₂ moieties [166] etc. As an alternative to using a linker, direct linkage can be used. Direct linkages to the protein may comprise oxidation of the polysaccharide followed by reductive amination with the protein, as described in, for example, references 167 and 168.

A process involving the introduction of amino groups into the saccharide (e.g. by replacing terminal ═O groups with —NH₂) followed by derivatisation with an adipic diester (e.g. adipic acid N-hydroxysuccinimido diester) and reaction with carrier protein is preferred. Another preferred reaction uses CDAP activation with a protein D carrier e.g. for MenA or MenC.

Non-Meningococcal Antigens

A composition may include a non-meningococcal antigen e.g. from a non-meningococcal pathogen, such as a bacterium or virus. Thus a composition may include one or more of the following further antigens:
a saccharide antigen from Streptococcus pneumoniae
an antigen from hepatitis A virus, such as inactivated virus
an antigen from hepatitis B virus, such as the surface and/or core antigens
a diphtheria antigen, such as a diphtheria toxoid
a tetanus antigen, such as a tetanus toxoid
an antigen from Bordetella pertussis, such as pertussis holotoxin (PT) and filamentous haemagglutinin (FHA) from B. pertussis, optionally also in combination with pertactin and/or agglutinogens 2 and 3
a saccharide antigen from Haemophilus influenzae B
polio antigen(s) such as IPV.
an antigen from Moraxella catarrhalis
a protein and/or saccharide antigen from Streptococcus agalactiae (group B streptococcus)
an antigen from Streptococcus pyogenes (group A streptococcus)
an antigen from Staphylococcus aureus

The composition may comprise one or more of these further antigens.

Toxic protein antigens may be detoxified where necessary (e.g. detoxification of pertussis toxin by chemical and/or genetic means).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred.

Saccharide antigens are preferably in the form of conjugates. Carrier proteins for the conjugates are discussed in more detail above.

Antigens in the composition will typically be present at a concentration of at least 1 μg/ml each. In general, the concentration of any given antigen will be sufficient to elicit an immune response against that antigen.

Immunisation

In addition to providing immunogenic compositions as described above, the invention also provides a method for raising an antibody response in a mammal, comprising administering an immunogenic composition of the invention to the mammal. The antibody response is preferably a protective and/or bactericidal antibody response. The invention also provides compositions of the invention for use in such methods.

The invention also provides a method for protecting a mammal against a Neisserial (e.g. meningococcal) infection and/or disease (e.g. against meningococcal meningitis), comprising administering to the mammal an immunogenic composition of the invention.

The invention provides compositions of the invention for use as medicaments (e.g. as immunogenic compositions or as vaccines). It also provides the use of vesicles of the invention in the manufacture of a medicament for preventing Neisserial (e.g. meningococcal) infection in a mammal.

The mammal is preferably a human. The human may be an adult or, preferably, a child. Where the vaccine is for prophylactic use, the human is preferably a child (e.g. a toddler or infant); where the vaccine is for therapeutic use, the human is preferably an adult. A vaccine intended for children may also be administered to adults e.g. to assess safety, dosage, immunogenicity, etc.

The uses and methods are particularly useful for preventing/treating diseases including, but not limited to, meningitis (particularly bacterial, such as meningococcal, meningitis) and bacteremia.

Efficacy of therapeutic treatment can be tested by monitoring Neisserial infection after administration of the composition of the invention. Efficacy of prophylactic treatment can be tested by monitoring immune responses against fHBP or other antigens after administration of the composition. Immunogenicity of compositions of the invention can be determined by administering them to test subjects (e.g. children 12-16 months age, or animal models [169]) and then determining standard parameters including serum bactericidal antibodies (SBA) and ELISA titres (GMT). These immune responses will generally be determined around 4 weeks after administration of the composition, and compared to values determined before administration of the composition. A SBA increase of at least 4-fold or 8-fold is preferred. Where more than one dose of the composition is administered, more than one post-administration determination may be made.

Preferred compositions of the invention can confer an antibody titre in a patient that is superior to the criterion for seroprotection for each antigenic component for an acceptable percentage of human subjects. Antigens with an associated antibody titre above which a host is considered to be seroconverted against the antigen are well known, and such titres are published by organisations such as WHO. Preferably more than 80% of a statistically significant sample of subjects is seroconverted, more preferably more than 90%, still more preferably more than 93% and most preferably 96-100%.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is about 0.5 ml.

The invention may be used to elicit systemic and/or mucosal immunity.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

Bactericidal Responses

Preferred immunogenic compositions can elicit antibody responses that are bactericidal against meningococci. Bactericidal antibody responses are conveniently measured in mice and are a standard indicator of vaccine efficacy [e.g. see end-note 14 of reference 170]. Compositions of the invention can preferably elicit an antibody response which is bactericidal against at least one N. meningitidis strain from each of at least two of the following three groups of strains:
(I) MC58, gb185(=M01-240185), m4030, m2197, m2937, iss1001, NZ394/98, 67/00, 93/114, bz198, m1390, nge28, lnp17592, 00-241341, f6124, 205900, m198/172, bz133, gb149(=M01-240149), nm008, nm092, 30/00, 39/99, 72/00, 95330, bz169, bz83, cu385, h44/76, m1590, m2934, m2969, m3370, m4215, m4318, n44/89, 14847.
(II) 961-5945, 2996, 96217, 312294, 11327, a22, gb013(=M01-240013), e32, m1090, m4287, 860800, 599, 95N477, 90-18311, c11, m986, m2671, 1000, m1096, m3279, bz232, dk353, m3697, ngh38, L93/4286.
(III) M1239, 16889, gb355(=M01-240355), m3369, m3813, ngp165.

For example, a composition may elicit a bactericidal response effective against two or three of serogroup B N. meningitidis strains MC58, 961-5945 and M1239.

Compositions can preferably elicit an antibody response which is bactericidal against at least 50% of clinically-relevant meningococcal serogroup B strains (e.g. 60%, 70%, 80%, 90%, 95% or more). The composition may elicit an antibody response which is bactericidal against strains of serogroup B N. meningitidis and strains of at least one (e.g. 1, 2, 3, 4) of serogroups A, C, W135 and Y. The composition may elicit an antibody response which is bactericidal against strains of N. gonorrhoeae and/or N. cinerea. The composition may elicit a response which is bactericidal against strains from at least two of the three main branches of the dendrogram shown in FIG. 5 of reference 2.

Compositions may elicit an antibody response which is bactericidal against N. meningitidis strains in at least 2 (e.g. 2, 3, 4, 5, 6, 7) of hypervirulent lineages ET-37, ET-5, cluster A4, lineage 3, subgroup 1, subgroup III, and subgroup IV-1 [171,172]. Compositions may additionally induce bactericidal antibody responses against one or more hyperinvasive lineages.

Compositions may elicit an antibody response which is bactericidal against N. meningitidis strains in at least at least 2 (e.g. 2, 3, 4, 5, 6, 7) of the following multilocus sequence types: ST1, ST4, ST5, ST8, ST11, ST32 and ST41 [173]. The composition may also elicit an antibody response which is bactericidal against ST44 strains.

The composition need not induce bactericidal antibodies against each and every MenB strain within the specified lineages or MLST; rather, for any given group of four of more strains of serogroup B meningococcus within a particular hypervirulent lineage or MLST, the antibodies induced by the composition are preferably bactericidal against at least 50% (e.g. 60%, 70%, 80%, 90% or more) of the group. Preferred groups of strains will include strains isolated in at least four of the following countries: GB, AU, CA, NO, IT, US, NZ, NL, BR, and CU. The serum preferably has a bactericidal titre of at least 1024 (e.g. 2¹⁰, 2¹¹, 2¹², 2¹³, 2¹⁴, 2¹⁵, 2¹⁶, 2¹⁷, 2¹⁸ or higher, preferably at least 2¹⁴) i.e. the serum is able to kill at least 50% of test bacteria of a particular strain when diluted 1:1024 e.g. as described in end-note 14 of reference 170. Preferred compositions can elicit an antibody response in mice that remains bactericidal even when the serum is diluted 1:4096 or further.

General

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

“Sequence identity” is preferably determined by the Smith-Waterman homology search algorithm as implemented in the MPSRCH program (Oxford Molecular), using an affine gap search with parameters gap open penalty=12 and gap extension penalty−1.

After serogroup, meningococcal classification includes serotype, serosubtype and then immunotype, and the standard nomenclature lists serogroup, serotype, serosubtype, and immunotype, each separated by a colon e.g. B:4:P1.15:L3,7,9. Within serogroup B, some lineages cause disease often (hyperinvasive), some lineages cause more severe forms of disease than others (hypervirulent), and others rarely cause disease at all. Seven hypervirulent lineages are recognised, namely subgroups I, III and IV-1, ET-5 complex, ET-37 complex, A4 cluster and lineage 3. These have been defined by multilocus enzyme electrophoresis (MLEE), but multilocus sequence typing (MLST) has also been used to classify meningococci [ref. 173]. The four main hypervirulent clusters are ST32, ST44, ST8 and ST11 complexes.

### BRIEF DESCRIPTION OF THE DRAWINGS

FIG. 1 shows the genome sequence (SEQ ID NO: 17) around the start codon of the nmb1869 gene.

FIG. 2 shows the genome sequence (SEQ ID NO: 18) around the start codon of the fhbp gene.

FIG. 3 shows a northern blot of short and long fhbp transcripts in various strains.

FIG. 4 shows a western blot of three different proteins in various strains.

### MODES FOR CARRYING OUT THE INVENTION

Further information is available from reference 174.

Analysis of the fhbp Gene Locus in MC58 Strain of Neisseria meningitidis

The fhbp gene is flanked by the nmb1869 (fructose-bisphosphate aldolase) and nmb1871 genes. Transcriptional terminator analysis revealed a typical stem-loop of a Rho-independent terminator 11 nt downstream of the fhbp gene. There is an intergenic region of 157 bps between the nmb1869 and the GTG starting codon of the fhbp gene. In this intergenic region, 20 nucleotides downstream of the nmb1869 gene, there is another putative Rho-independent transcriptional terminator. These initial observations of the locus suggest that the fHBP gene may be transcribed as a single gene and is not member of an operon.

RT-PCR analysis of total RNA from MC58 resulted in an amplification product across the upstream intergenic region but not the downstream intergenic region, suggesting that fhbp may be transcribed with the upstream nmb1869 gene.

Northern blot analysis on total RNA from the MC58 wild-type strain, NMB 1869 null mutant (Δnmb1869) and fhbp null mutant (Δfhbp) strains revealed a long transcript, >2000 nt, detected by both fhbp and nmb1869 probes in the wild-type strain but absent in both mutant strains. The estimated size of this transcript corresponds well to the predicted size of a bicistronic message and confirms the co-transcription of nmb1869 and fhbp genes. A shorter fhbp-specific mRNA of just under 1000 nt was also detected in the wild-type and in the Δnmb1869 mutant strain, suggesting the presence of a fhbp monocistronic transcript. In addition the presence of this shorter transcript in the Δnmb1869 mutant, where transcription of the bicistronic message has been eliminated, indicates that the short fhbp transcript is due to the transcription of the fhbp gene driven from its own dedicated promoter and not as a result of processing of the longer transcript.

In addition, the nmb1869 probe detected a smaller nmb1869-specific transcript of ˜1100 nt in the wild-type strain and in the fhbp null mutant, indicating that a monocistronic transcript of the nmb1869 upstream gene was also produced. Taken together these results suggest that two different promoters drive the synthesis of three separate mRNA transcripts of the nmb1869 and fhbp locus. The nmb1869 and fhbp genes are transcribed on monocistronic transcripts from their dedicated promoters but are also are co-transcribed on a bicistronic transcript driven by a promoter upstream of nmb1869. The longer bicistronic transcript probably results from inefficient termination that leads to read-through of the transcriptional terminator downstream of NMB1869.

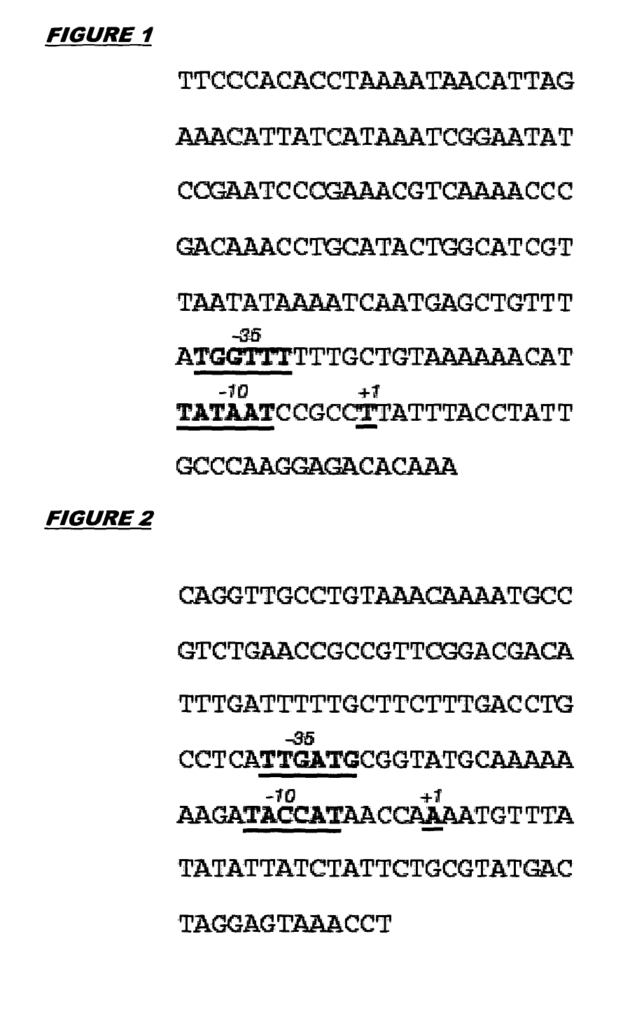

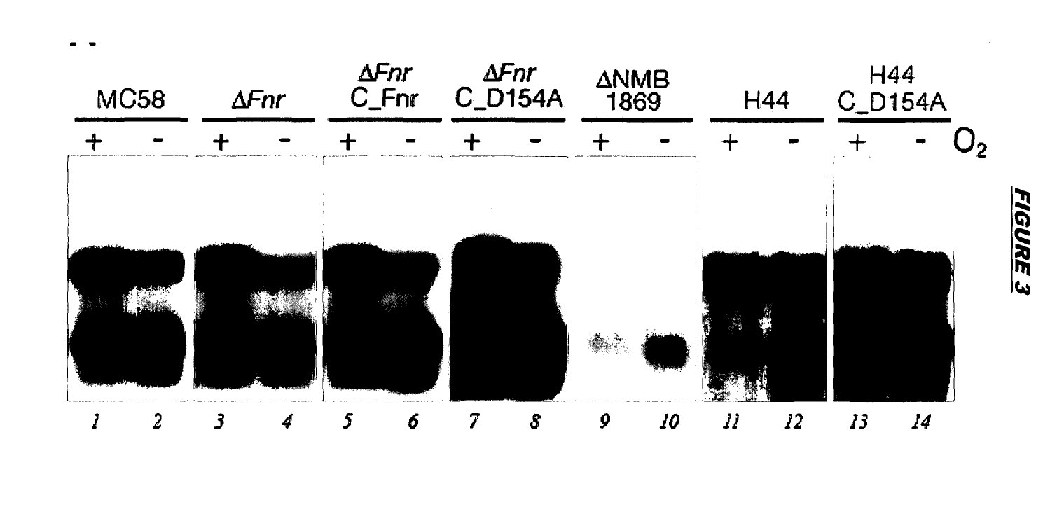

Primer extension of total RNA extracted from N. meningitidis cultures grown to mid-log phase was used to define the start point of the mRNAs. A nmb1869-specific primer was hybridized to total RNA from MC58 and elongated with reverse transcriptase. The major elongated product maps the 5′ end of the nmb1869-transcripts to a position 29 nucleotides upstream of its start codon (FIG. 1). Primer extension with a fhbp-specific primer was also performed with total RNA from MC58 and from the Δ1869 mutant, and this work mapped the start of the fhbp monocistronic transcript to a position 45 nucleotides upstream of its start codon of fhbp (FIG. 2). The nucleotide sequences in each case upstream of the elongated primers show the presence of elements similar to the −10 and the −35 hexamers of σ70-dependent promoters from E. coli (FIGS. 1 & 2). These sequences should define the N. meningitidis P_{nmb1869} and P_{fhbp} promoters.

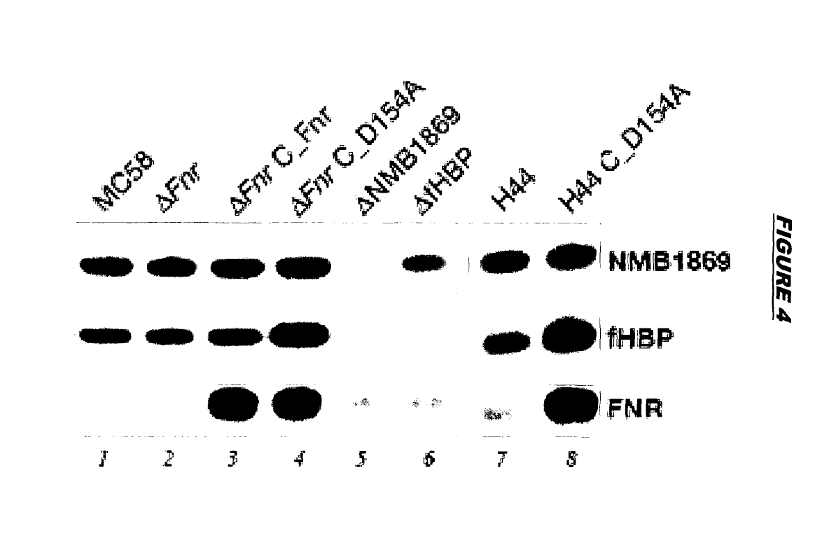

After identification of the two promoters driving the synthesis of three mRNAs, their regulation mechanisms were explored. Expression of the fhbp gene was investigated under different oxygen conditions. Total RNA was extracted from the wild-type strain and the fnr null mutant grown to mid-log phase and then exposed to microaerobic conditions (+) or oxygen-limitation conditions (−) for 30 minutes. Northern blot analysis was carried out to analyze the levels of the two fHBP transcripts (FIG. 3). The monocistronic transcript were up-regulated during oxygen limitation in the wild-type (lane 2 vs. lane 1) but not in the fnr null mutant (lanes 3 & 4) indicating that FNR mediates the induction under oxygen-limitation. For confirmation of FNR-dependent regulation, a mutant strain expressing a single copy of the fnr gene in a heterologous location on the chromosome was used. In this Δfnr_C strain the up-regulation of the fhbp monocistronic mRNA was restored under oxygen-limitation (lanes 5 & 6). In addition, the longer bicistronic transcript was expressed at a lower level during oxygen limiting conditions but it seemed not to be an FNR-dependent regulation.

Constitutively Active Meningococcal FNR

Although reference 29 had identified the fhbp gene as a possible member of the FNR regulon, it had provided no experimental confirmation and had not realised the existence of two distinct fhbp transcripts, only one of which is FNR-activated. To further confirm the FNR-activated activity, a constitutively active form of FNR protein was created.

It is known that FNR from E. coli can be modified such that its [4Fe-4S] cluster is O₂-stable. One mutation which achieves this stability is D148A in which Asp-148 (e.g. in SEQ ID NO: 6, E. coli strain CFT073) is mutated to Ala. This single amino acidic substitution in the putative dimerization domain of FNR, resulted in a constitutively active protein which could function as an transcriptional activator also in the presence of oxygen [175].

The E. coli sequence of SEQ ID NO: 6 aligns with meningococcal sequence (SEQ ID NO: 4):

| | | |
|---|---|---|
| SEQID4 | MASHNTTHQMKT------LCSSCSLRELCLPVGLLPNELSQLDAVIRQSRRLKKGEYLFC | 54 |
| SEQID6 | MIPEKRIIRRIQSGGCAIHCQDCSISQLCIPFTLNEHELDQLDNIIERKKPIQKGQTLFK | 60 |
| | :..:. ::::: *..**: :**:*. * :**.*** :*.:.: ::**: ** | |
| SEQID4 | VGEAFTSLFAIRSGFFKTTVASQDGRDQVTGFFMSGELIGMDGICSHVHSCDAVALEDSE | 114 |
| SEQID6 | AGDELKSLYAIRSGTIKSYTITEQGDEQITGFHLAGDLVGFDAIGSGHHPSFAQALETSM | 120 |
| | .*: :.**:***** :*: . :::* :*:***.::*:*:*:*.* * *.. * *** * | |
| SEQID4 | VCELPFTHIEELGQNIPSLRTHFFRMMSREIVRDQGVMLLLGNMRAEERIAAFLLNLSQR | 174 |
| SEQID6 | VCEIPFETLDDLSGKMPNLRQQMMRLMSGEIKGDQDMILLLSKKNAEERLAAFIYNLSRR | 180 |
| | ***:** :::*. ::*.** :::*:** ** **.::***.: .****:***: ***:* | |
| SEQID4 | LYSRGFAANDFILRMSREEIGSYLGLKLETVSRTLSKFHQEGLISVEHKHIKILNLQVLK | 234 |
| SEQID6 | FAQRGFSPREFRLTMTRGDIGNYLGLTVETISRLLGRFQKSGMLAVKGKYITIENNDALA | 240 |
| | : .***:..:* * *:* :**.****.:**:** *.:*::.*:::*: *:*.* * :.* | |
| SEQID4 | KMVSGCSHAI | 244 |
| SEQID6 | QLAGHTRNVA | 250 |
| | ::.. :. | |

Despite the overall low identity (40%) between the two sequences, E. coli residue D154 (underlined) is also present in the meningococcal sequence at residue 148.

Site-directed mutagenesis was used to replace the codon encoding Asp-148 of the meningococcal sequence and substitute it with the GCC alanine codon. This modified gene, and its encoded protein, is referred to hereafter as “fnrD148A”.

A Δfnr null mutant of meningococcus was produced by replacing the entire coding sequence with an erythromycin resistance cassette [29]. For complementation of the fnr null mutant, a wild-type fnr or a D148A mutant fnr gene, under the control of a Ptac promoter, was integrated into the chromosome of Afnr between the converging ORFs NMB 1428 and NMB 1429, through the transformation of the Δfnr strain with the pCompInd-fnr or pCompInd-fnrD148A, respectively. The pCompInd-fnr is a derivative plasmid of pCompInd in which the wild-type fnr gene was amplified from the MC58 genome and cloned as a 732 bp NdeI/NsiI fragment downstream of the Ptac promoter. The pCompInd-fnrD148A plasmid is a derivative of pCompInd-fnr encoding fnrD148A. The mutation was introduced in the pCompInd-fnr using the QuickChange™ kit (StratageneTM). The Δfnr strain was transformed with the pCompInd-fnr or pCompInd-fnrD148A plasmids. Furthermore to generate recombinant strains expressing a FnrD148A protein from an integrated copy of the mutant gene, the pCompInd-fnrD148A plasmid was transformed into meningococcal isolates H44/76, 4243, F6124, M6190, LNP17592, M01-240345, NM117, LNP17094, B3937, M01-240013, M3153, 5/99, BZ232, 1000, OX99.30304, generating derivative versions of each strain.

For transformation of naturally competent N. meningitidis, four or five single colonies of a freshly grown overnight culture were re-suspended in 20 μl of PBS, spotted onto GC agar plates to which 5 to 10 μg of linearized plasmid DNA was added, allowed to dry and incubated for 6 to 8 h at 37° C. Transformants were then selected on plates containing the appropriate antibiotic, and single colonies were re-streaked on selective media for further analysis. Single colonies were resuspended in 50 μl of PBS and placed in a boiling water-bath for 5 min and centrifuged in a benchtop centrifuge for 5 minutes at maximum speed. One μl of the sample was used as template for PCR analysis for correct double crossover transformants.

Northern blot analysis using total RNA from a FNR knockout strain, complemented with the mutant gene (Δfnr_CD148A), grown either during microaerobic or oxygen-limiting conditions, showed that the mutant FNR protein was able to promote transcription of the fhbp monocistronic mRNA even in the presence of oxygen (FIG. 3, lanes 7 & 8). Thus the mutant FNR drives transcription in an oxygen-independent manner. Knocking out the upstream nmb1869 gene, thereby abolishing the synthesis of the bicistronic RNA messenger, did not affect the FNR-oxygen-dependent regulation of the monocistronic transcript (FIG. 3, lanes 9 & 10). Taken together these data show that transcription of fhbp is induced under oxygen limitation by a dedicated FNR-activated promoter.

Transcription and the regulation of the fhbp gene was also studied in strain H44/76. This work also showed two fhbp transcripts, and confirmed that the fhbp monocistronic mRNA was upregulated in response to oxygen limitation in the wild-type strain and also by the expression of the constitutively active FNR mutant protein (FIG. 3, lanes 11-14).

Western blot analysis was used to correlate the transcriptional regulation by FNR to overall protein levels in all strains. Total protein extracts were prepared from freshly grown overnight plate cultures under micro-aerobic conditions and immunoblotted with specific antibodies raised against the NMB1869, fHBP and FNR proteins. As shown in FIG. 4, fHBP expression was significantly increased in the Δfnr_CD148A and H44/76_CD148A strains expressing the constitutively active form of FNR (lanes 4 & 8), correlating with the Northern results under microaerobic conditions.

Furthermore, in the recombinant strains, there is an over-expression of the respective FNR protein alleles expressed from the heterologous P_{tac} promoter compared to FNR expression in the wild-type strain, but only the D148A mutant induced over-expression of fHBP. These data strongly support the importance of FNR activity, rather than its high expression, in promoting fHBP expression.

Genes encoding wild-type and mutant FNR proteins (the fnr and fnrD148A genes) were cloned into pET15b expression vector for recombinant expression in E. coli. The proteins were expressed and purified by Ni²⁺-affinity chromatography by virtue of an N-terminus histidine tag.

The in vitro binding activity of both recombinant proteins to the aniA promoter was tested. This promoter has been well characterized through DNA microarray and DNA binding studies in N. meningitidis and N. gonorrhoeae and is under the direct control of FNR during oxygen limitation [28, 29, 176]. A specific probe containing the aniA promoter of MC58 was incubated with increasing concentrations of the recombinant proteins and submitted to DNase I digestion. Addition of 13 nM FNRD148A protein resulted in complete protection of the DNA region spanning −30 to −50 with respect to the transcriptional start site and containing the aniA predicted FNR-box consensus. Under these conditions, however, the wild-type protein did not result in protection. Thus the mutant is constitutively active for DNA-binding under aerobic conditions and binds to the predicted FNR-box.

Addition of 1 μM of FnrD148A protected nucleotides spanning from positions −28 to −50 with respect to the transcriptional start site of P_{fhbp}, therefore, overlapping the −35 promoter element. Analysis of the promoter sequence revealed the presence of a putative FNR-box, TTGAC-N₄-CTCAT (SEQ ID NO: 16) just overlapping the −35 hexamer. This sequence differs by three nucleotides from the E. coli FNR box consensus (SEQ ID NO: 19). These data indicate that FNR binds the fhbp promoter region to promote transcription and expression of fHBP protein.

Investigations in Multiple Strains

FNR-dependent regulation of fHBP protein expression was also studied in other meningococcal strains from geographically diverse origins and representing the main clonal complexes associated with disease. A preliminary Western blot analysis was performed on strains in different fHBP families. The fHBP antigen was expressed by all of the strains but, as previously noted [2], the level of expression varied between strains. Isogenic mutant strains expressing the constitutively active FNR were made using the same construct which had been used to create the MC58 ΔfnrC_D148A strain. Western blot analysis was carried out on the obtained transformants and their respective wild-type. In these mutant strains the endogenous fnr gene was not inactivated.

Transformed strains expressed the FNR protein at a higher level compared to the respective wild-types, confirming the success of transformation. Moreover, the recombinant strains also over-expressed fHBP protein. The only exception was represented by the NM117 strain. Although it over-expressed FNR, it but did not significantly overexpress fHBP. The P_{fhbp} promoter was sequenced and, although the FNR-box was perfectly conserved, the −10 promoter element had 2 mutations with respect to the MC58 sequence, exhibiting a TACCGC sequence (SEQ ID NO: 15) which is unlikely to act as an efficient −10 element.

Taken together, these results show that the FNR-dependent regulation of the fhbp gene is not restricted to the MC58 and H44176 strains.

Vesicle Production

As disclosed above, strains encodingfhbp from its natural promoter(s) over-express fHBP when they express a constitutively-active form of FNR. Vesicles prepared from these strains, ideally without the use of detergent [25,44], are thus enriched for fHBP relative to vesicles prepared from the corresponding wild-type strain. These vesicles can be used for generating anti-meningococcal immunity. Bivalent or trivalent mixtures of such vesicles, each having fHBP from a different family, can be used to improve the spectrum of coverage. In other embodiments, the meningococcus expressing a constitutively-active FNR is engineered to express two or three fHBP variants such that the vesicles from that strain are already bivalent or trivalent for fHBP. The different variants can be expressed separately, from exogenous genes integrated at different points in the chromosome, but each under the control of a FNR-activated promoter.

It will be understood that the invention is described above by way of example only and modifications may be made whilst remaining within the scope and spirit of the invention.

### REFERENCES

[1] WO99/57280.

[2] Masignani et al. (2003) J Exp Med 197:789-799.

[3] Welsch et al. (2004) J Immunol 172:5605-15.

[4] Hou et al. (2005) J Infect Dis 192(4):580-90.

[5] WO03/063766.

[6] Fletcher et al. (2004) Infect Immun 72:2088-2100.

[7] Zhu et al. (2005) Infect Immun 73(10):6838-45.

[8] WO2004/048404.

[9] WO2006/081259.

[10] WO01/09350.

[11] European patent 0449958.

[12] EP-A-0996712.

[13] EP-A-0680512.

[14] WO98/56901.

[15] Claassen et al. (1996) 14(10):1001-8.

[16] WO99/10497.

[17] Steeghs et al. (2001) The EMBO Journal 20:6937-6945.

[18] WO01/52885.

[19] WO00/25811.

[20] WO01/09350.

[21] WO02/09746.

[22] WO02/062378.

[23] WO2004/014417.

[24] WO2006/046143.

[25] WO2008/038889.

[26] Koeberling et al. (2009) Clin Vaccine Immunol 16:156-62.

[27] Bates et al. (1995) J Bacteriol 177:3972-8.

[28] Overton et al. (2003) J Bacteriol 185:4734-7.

[29] Bartolini et al. (2006) Mol Microbiol 60:963-72.

[30] WO2009/104097.

[31] WO2006/024954.

[32] WO2007/060548.

[33] WO2009/114485

[34] WO01/64920.

[35] WO03/020756.

[36] WO2004/048404.

[37] WO2008/079372.

[38] U.S. provisional application 61/237,576.

[39] WO02/09643.

[40] Katial et al. (2002) Infect. Immun. 70:702-707.

[41] U.S. Pat. No. 6,180,111.

[42] WO01/34642.

[43] WO02/062378.

[44] WO2004/019977.

[45] European patent 0011243.

[46] Fredriksen et al. (1991) NIPH Ann. 14(2):67-80.

[47] WO01/91788.

[48] WO2005/004908.

[49] Gennaro (2000) Remington: The Science and Practice of Pharmacy. 20th edition, ISBN: 0683306472.

[50] WO03/009869.

[51] WO90/14837.

[52] Podda & Del Giudice (2003) Expert Rev Vaccines 2:197-203.

[53] Podda (2001) Vaccine 19: 2673-2680.

[54] Vaccine Design: The Subunit and Adjuvant Approach (eds. Powell & Newman) Plenum Press 1995 (ISBN 0-306-44867-X).

[55] Vaccine Adjuvants: Preparation Methods and Research Protocols (Volume 42 of Methods in Molecular Medicine series). ISBN: 1-59259-083-7. Ed. O'Hagan.

[56] Allison & Byars (1992) Res Immunol 143:519-25.

[57] Hariharan et al. (1995) Cancer Res 55:3486-9.

[58] US-2007/014805.

[59] WO95/11700.

[60] U.S. Pat. No. 6,080,725.

[61] WO2006/113373.

[62] WO2005/097181.

[63] U.S. Pat. No. 5,057,540.

[64] WO96/33739.

[65] EP-A-0109942.

[66] WO96/11711.

[67] WO00/07621.

[68] Barr et al. (1998) Advanced Drug Delivery Reviews 32:247-271.

[69] Sjolanderet et al. (1998) Advanced Drug Delivery Reviews 32:321-338.

[70] EP-A-0689454.

[71] Johnson et al. (1999) Bioorg Med Chem Lett 9:2273-2278.

[72] Evans et al. (2003) Expert Rev Vaccines 2:219-229.

[73] Meraldi et al. (2003) Vaccine 21:2485-2491.

[74] Pajak et al. (2003) Vaccine 21:836-842.

[75] Kandimalla et al. (2003) Nucleic Acids Research 31:2393-2400.

[76] WO02/26757.

[77] WO99/62923.

[78] Krieg (2003) Nature Medicine 9:831-835.

[79] McCluskie et al. (2002) FEMS Immunology and Medical Microbiology 32:179-185.

[80] WO98/40100.

[81] U.S. Pat. No. 6,207,646.

[82] U.S. Pat. No. 6,239,116.

[83] U.S. Pat. No. 6,429,199.

[84] Kandimalla et al. (2003) Biochemical Society Transactions 31 (part 3):654-658.

[85] Blackwell et al. (2003) J Immunol 170:4061-4068.

[86] Krieg (2002) Trends Immunol 23:64-65.

[87] WO01/95935.

[88] Kandimalla et al. (2003) BBRC 306:948-953.

[89] Bhagat et al. (2003) BBRC 300:853-861.

[90] WO03/035836.

[91] Schellack et al. (2006) Vaccine 24:5461-72.

[92] Lingnau et al. (2007) Expert Rev Vaccines 6:741-6.

[93] WO2004/084938.

[94] WO95/17211.

[95] WO98/42375.

[96] Beignon et al. (2002) Infect Immun 70:3012-3019.

[97] Pizza et al. (2001) Vaccine 19:2534-2541.

[98] Pizza et al. (2000) Int J Med Microbiol 290:455-461.

[99] Scharton-Kersten et al. (2000) Infect Immun 68:5306-5313.

[100] Ryan et al. (1999) Infect Immun 67:6270-6280.

[101] Partidos et al. (1999) Immunol Lett 67:209-216.

[102] Peppoloni et al. (2003) Expert Rev Vaccines 2:285-293.

[103] Pine et al. (2002) J Control Release 85:263-270.

[104] Tebbey et al. (2000) Vaccine 18:2723-34.

[105] Domenighini et al. (1995) Mol Microbiol 15:1165-1167.

[106] WO99/40936.

[107] WO99/44636.

[108] Singh et al] (2001) J Cont Release 70:267-276.

[109] WO99/27960.

[110] U.S. Pat. No. 6,090,406.

[111] U.S. Pat. No. 5,916,588.

[112] EP-A-0626169.

[113] Stanley (2002) Clin Exp Dermatol 27:571-577.

[114] Jones (2003) Curr Opin Investig Drugs 4:214-218.

[115] WO99/11241.

[116] WO94/00153.

[117] WO98/57659.

[118] European patent applications 0835318, 0735898 and 0761231.

[119] Giuliani et al. (2006) Proc Natl Acad Sci USA. 103:10834-9.

[120] WO2004/032958.

[121] Tettelin et al. (2000) Science 287:1809-1815.

[122] WO00/66741.

[123] WO99/57280

[124] Martin et al. (1997) J Exp Med 185(7):1173-83.

[125] WO96/29412.

[126] Perkins-Balding et al. (2003) Microbiology 149:3423-35.

[127] WO01/55182.

[128] WO01/38350.

[129] WO00/23595.

[130] Tettelin et al. (2000) Science 287:1809-1815.

[131] Jones (2001) Curr Opin Investig Drugs 2:47-49.

[132] Ravenscroft et al. (1999) Vaccine 17:2802-2816.

[133] WO03/080678.

[134] Research Disclosure, 453077 (January 2002).

[135] EP-A-0372501.

[136] EP-A-0378881.

[137] EP-A-0427347.

[138] WO93/17712.

[139] WO94/03208.

[140] WO98/58668.

[141] EP-A-0471177.

[142] WO91/01146.

[143] Falugi et al. (2001) Eur J Immunol 31:3816-3824.

[144] Baraldo et al. (2004) Infect Immun 72(8):4884-7.

[145] EP-A-0594610.

[146] Ruan et al. (1990) J Immunol 145:3379-3384.

[147] WO00/56360.

[148] Kuo et al. (1995) Infect Immun 63:2706-13.

[149] Michon etal. (1998) Vaccine. 16:1732-41.

[150] WO02/091998.

[151] WO01/72337.

[152] WO00/61761.

[153] WO00/33882

[154] Lees et al. (1996) Vaccine 14:190-198.

[155] WO95/08348.

[156] U.S. Pat. No. 4,882,317

[157] U.S. Pat. No. 4,695,624

[158] Porro et al. (1985) Mol Immunol 22:907-919.s

[159] EP-A-0208375

[160] WO00/10599

[161] Gever et al. Med. Microbiol. Immunol, 165: 171-288 (1979).

[162] U.S. Pat. No. 4,057,685.

[163] U.S. Pat. Nos. 4,673,574; 4,761,283; 4,808,700.

[164] U.S. Pat. No. 4,459,286.

[165] U.S. Pat. No. 4,965,338

[166] U.S. Pat. No. 4,663,160.

[167] U.S. Pat. No. 4,761,283

[168] U.S. Pat. No. 4,356,170

[169] WO01/30390.

[170] Pizza et al. (2000) Science 287:1816-1820.

[171] Achtman (1995) Global epidemiology of meningococcal disease. Pages 159-175 of Meningococcal disease (ed. Cartwight). ISBN: 0-471-95259-1.

[172] Caugant (1998) APMIS 106:505-525.

[173] Maiden et al. (1998) Proc. Natl. Acad. Sci. USA 95:3140-3145.

[174] Oriente et al. (2010) J BacterioL 192(3):691-701.

[175] Lazazzera et al. (1993) Genes Dev. 7(10):1993-2005.

[176] Lissenden et al. (2000) Mol Microbiol. 37(4):839-55.

1. A meningococcus which (a) has a gene whose transcription is under the control of a fumarate and nitrate reductase regulator (FNR)-activated promoter, and (b) expresses a constitutively active form of FNR.

2. The meningococcus of claim 1, wherein the gene whose transcription is under the control of a FNR-activated promoter is fhbp.

3. A process for preparing a mutant meningococcus, comprising (a) a step of modifying its endogenous fnr gene such that the encoded FNR protein is constitutively active, or (b) introducing a gene encoding a constitutively active form of FNR.

4. A process for preparing a proteoliposomic meningococcal vesicle, comprising a step of treating the meningococcus of claim 1 to disrupt its outer membrane, thereby forming vesicles therefrom which include protein components of the outer membrane.

5. The process of claim 4, wherein the proteoliposomic meningococcal vesicle includes fHBP.

6. The process of claim 4, including a further step of separating the vesicles from any living and/or whole bacteria.

7. A meningococcus which expresses a constitutively active form of FNR.

8. The meningococcus of claim 1, wherein the meningococcus does not express an active LpxL1 enzyme.

9. The meningococcus of claim 1, wherein the meningococcus is a hyperblebbing meningococcus.

10. A constitutively active form of meningococcus FNR.

11. A process for preparing an immunogenic composition comprising a step of formulating vesicles prepared by the process of claim 4 with: a pharmaceutically acceptable carrier; and/or with an immunological adjuvant; and/or with one or more further immunogenic components.

12. The meningococcus of claim 1, wherein the constitutively active form of FNR has mutation D148A.

13. Nucleic acid encoding a constitutively active form of meningococcus FNR.

14. A vector comprising the nucleic acid of claim 13.

15. A host cell including the vector of claim 14.
